(19) United States
(12) Patent Application Publication
(10) Pub. No.: US 2024/0404624 A1
(43) Pub. Date: Dec. 5, 2024

(54) STRUCTURAL VARIANT ALIGNMENT AND VARIANT CALLING BY UTILIZING A STRUCTURAL-VARIANT REFERENCE GENOME

(71) Applicant: Illumina, Inc., San Diego, CA (US)

(72) Inventors: Fan Zhang, San Diego, CA (US); Daniel Lee Cameron, Melbourne (AU); Michael Ruehle, Fort Worth, TX (US); Sean Truong, Castle Pines, CO (US); Sujai Chari, Burlingame, CA (US); Arun Subramaniyan, San Diego, CA (US); John Cooper Roddey, San Diego, CA (US)

(21) Appl. No.: 18/731,046

(22) Filed: May 31, 2024

Related U.S. Application Data
(60) Provisional application No. 63/505,361, filed on May 31, 2023.

(51) Int. Cl. G16B 20/00; G16B 30/10; G16B 40/20

(52) U.S. Cl. CPC G16B 20/00; G16B 30/10; G16B 40/20

(57) ABSTRACT

This disclosure describes methods, non-transitory computer-readable media, and systems that can (i) identify reads that align with at least some portion of alternative contiguous sequences representing structural variant haplotypes within a structural variant reference genome and (ii) generate a structural-variant-alignment tag within an alignment file for such read alignments to guide identifying candidate structural-variant locations. In addition to employing structural-variant-alignment tags, the disclosed systems identify read fragments that align or overlap with portions of alternate contiguous sequences representing an insertion (or other structural variant) and further masks such insertion-overlapping read fragments as part of an alignment file. When a read aligns completely within an insertion-representing alternate contiguous sequence, the disclosed system can mark the genomic coordinate corresponding to a primary contiguous sequence at which the insertion alternate contiguous sequence is lifted over and generates an unaligned read base indicator indicating that such an insertion-aligned nucleotide read is masked.

### CROSS-REFERENCE TO RELATED APPLICATIONS

This application claims priority to and the benefit of U.S. Provisional Patent Application No. 63/505,361 titled, “IMPROVING STRUCTURAL VARIANT ALIGNMENT AND VARIANT CALLING BY UTILIZING A STRUCTURAL-VARIANT REFERENCE GENOME,” filed on May 31, 2023, which is incorporated herein by reference in its entirety.

### BACKGROUND

In recent years, biotechnology firms and research institutions have improved hardware and software for sequencing nucleotides and determining nucleobase calls for genomic samples. For instance, some existing sequencing machines and sequencing-data-analysis software (together “existing sequencing systems”) predict individual nucleobases within sequences by using conventional Sanger sequencing or sequencing-by-synthesis (SBS) methods. When using SBS, existing sequencing systems can monitor many thousands of oligonucleotides being synthesized in parallel from templates to predict nucleobase calls for growing nucleotide reads. A camera in many existing sequencing systems captures images of irradiated fluorescent tags incorporated into oligonucleotides. After capturing such images, some existing sequencing systems determine nucleobase calls for nucleotide reads corresponding to the oligonucleotides and send base-call data to a computing device with sequencing-data-analysis software, which aligns nucleotide reads with a reference genome. Based on differences between the aligned nucleotide reads and the reference genome, existing sequencing systems (e.g., a variant caller) determines genotype calls for genomic regions and identifies variants of a genomic sample.

Despite these recent advances, existing sequencing systems often incorrectly align nucleotide reads with a reference genome's primary contiguous sequences that comprise nucleobase content similar to structural variants or, conversely, with alternate contiguous sequences representing structural variants with a liftover relationship relative to various primary contiguous sequences. For example, when nucleotide reads span both (i) an insertion of a threshold length, a deletion of a threshold length, or other structural variant represented by an alternate contiguous sequence and (ii) a portion of a reference genome's primary contiguous sequence, some existing sequencing systems map fragments of the nucleotide reads to multiple, different locations on the primary contiguous sequence, thereby producing confusing and inconsistent read pile-ups for read alignment. For instance, some existing sequencing systems map fragments of nucleotide reads to both correct and incorrect genomic regions along a primary contiguous sequence. When the fragments of a nucleotide read should map to an insertion of a threshold length, a deletion of a threshold length, or other structural variant—but the mapping software instead maps such read fragments to different genomic regions along a reference genome's primary contiguous sequence-existing sequencing systems fail to identify supporting evidence of a structural variant.

As just suggested, existing sequencing systems often lack a model that accurately distinguishes nucleotide reads that span from (i) primary contiguous sequences to (ii) alternative contiguous sequences representing structural variants. For instance, existing sequencing systems often fail to locally align nucleotide reads relative to a breakpoint on a primary contiguous sequence when the nucleotide reads align with alternate contiguous sequences representing structural variants. Consequently, existing sequencing systems frequently misidentify certain read alignments with relatively low mapping quality as noise or as incorrectly mapped to a primary contiguous sequence. As a further consequence, existing sequencing systems often cannot accurately align or map nucleotide reads with unique sequences mismatching a reference genome's primary contiguous sequence-when such nucleotide reads would best map to an insertion of a threshold length, a deletion of a threshold length, or other structural variant.

Due in part to inaccuracies of aligning or mapping read fragments, existing sequencing systems often determine false positive or false negative structural variant calls and other inaccurate variant calls. Indeed, some existing sequencing systems determine false positive structural variants—and false negative reference calls that should be structural variants—by lacking a model that accurately accounts for breakpoints for nucleotide read fragments corresponding to structural variants. By failing to accurately model nucleotide reads spanning a breakpoint that aligns with the portions of the primary contiguous sequence and portions of the alternative contiguous sequence, some existing sequencing systems incorrectly disregard a read alignment that correctly reflects a structural variant and fills in gaps indicative of an insertion or a threshold length or a deletion of a threshold length.

These, along with additional problems and issues exist in existing sequencing systems.

### BRIEF SUMMARY

This disclosure describes implementations of methods, non-transitory computer-readable media, and systems that can solve one or more of the foregoing (or other problems) in the art. For example, the disclosed systems can (i) identify reads that align with at least some portion of alternative contiguous sequences representing structural variant haplotypes within a structural variant reference genome and (ii) generate a structural-variant-alignment tag within an alignment file for such read alignments to guide one or both of identifying candidate structural-variant locations and calling variants. In addition to employing structural-variant-alignment tags, the disclosed system identifies nucleotide read fragments that align or overlap with portions of alternate contiguous sequences representing an insertion (or other structural variant) and further masks such insertion-overlapping read fragments as part of an alignment file. When a nucleotide read aligns completely within an alternate contiguous sequence representing an insertion as the relevant structural variant haplotype, in some cases, the disclosed system marks the genomic coordinate corresponding to a primary contiguous sequence at which the insertion alternate contiguous sequence is lifted over and generates an unaligned read-base indicator indicating that such an insertion-aligned nucleotide read is masked with respect to the marked genomic coordinate. As explained below, each of the structural-alignment-variant tag, insertion-overlap guided masking (or clipping), and insertion coordinate identifier facilitate more accurate mapping and variant calling with respect to existing sequencing systems.

Additional features and advantages of one or more implementations of the present disclosure are outlined in the description which follows, and in part will be obvious from the description, or may be learned by the practice of such example implementations.

### BRIEF DESCRIPTION OF THE DRA WINGS

The detailed description provides one or more implementations with additional specificity and detail through the use of the accompanying drawings, as briefly described below.

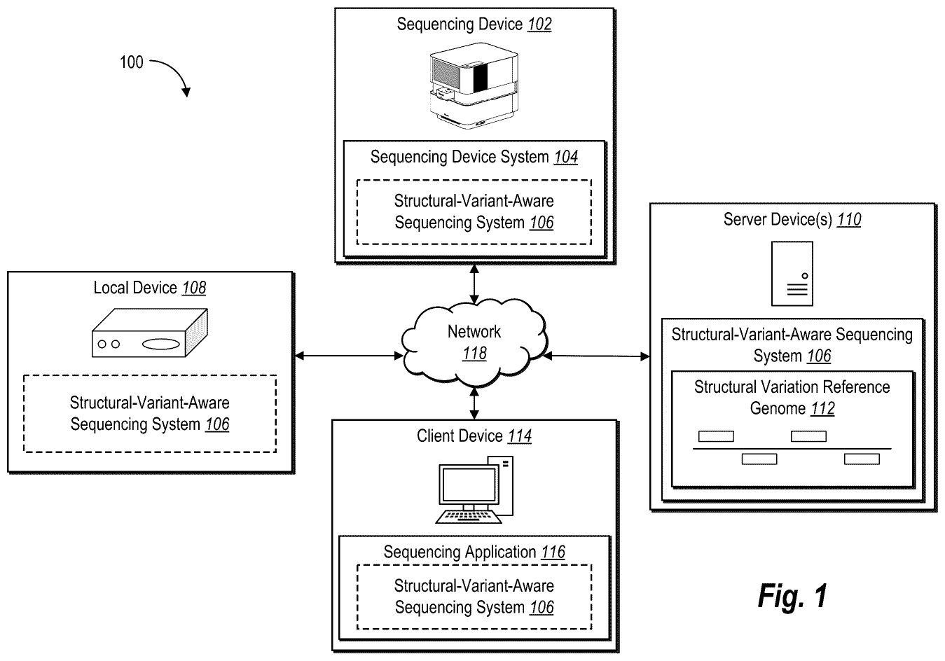

FIG. 1 illustrates a diagram of an environment in which a structural-variant-aware sequencing system can operate in accordance with one or more implementations.

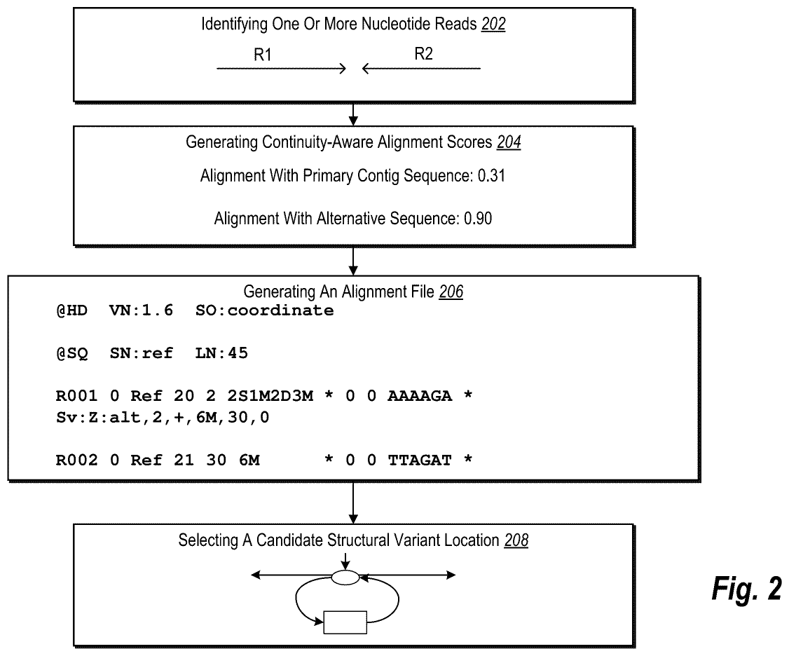

FIG. 2 illustrates an overview of the structural-variant-aware sequencing system generating a structural-variant-alignment tag for one or more nucleotide reads and selecting a candidate structural variant location based on the structural-variant-alignment tag in accordance with one or more implementations.

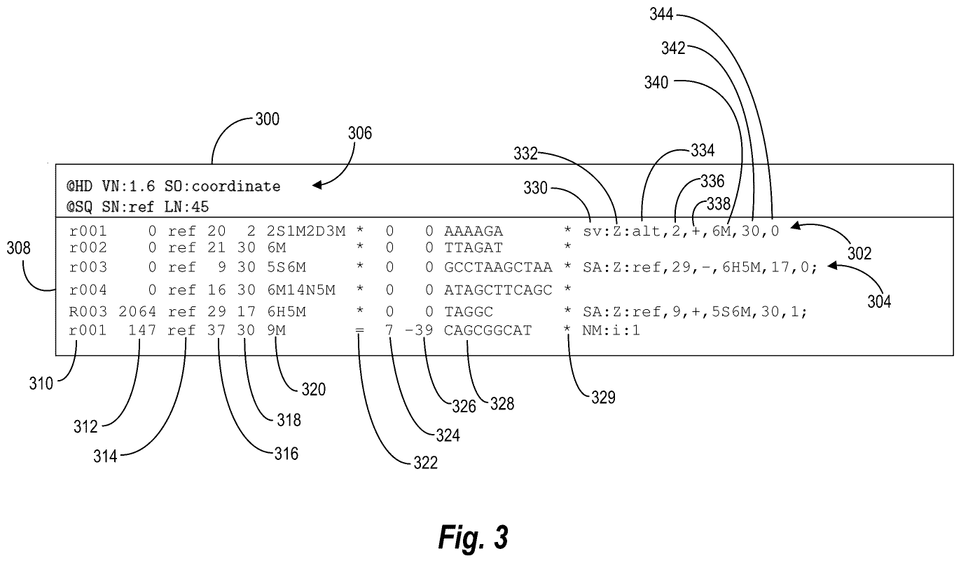

FIG. 3 illustrates the structural-variant-alignment tag indicating the alignment of one or more nucleotide reads with at least a part of the alternate contiguous sequence in accordance with one or more implementations.

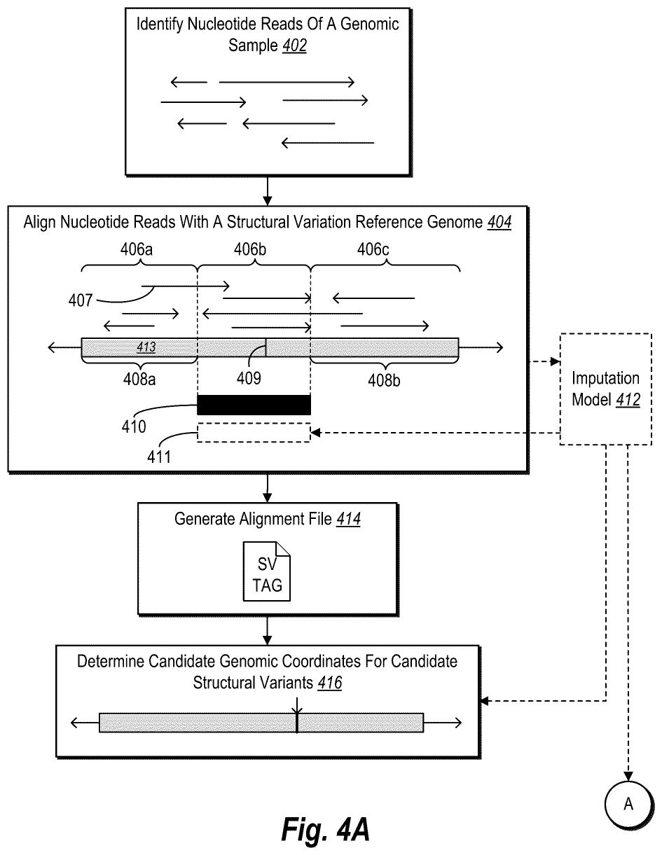

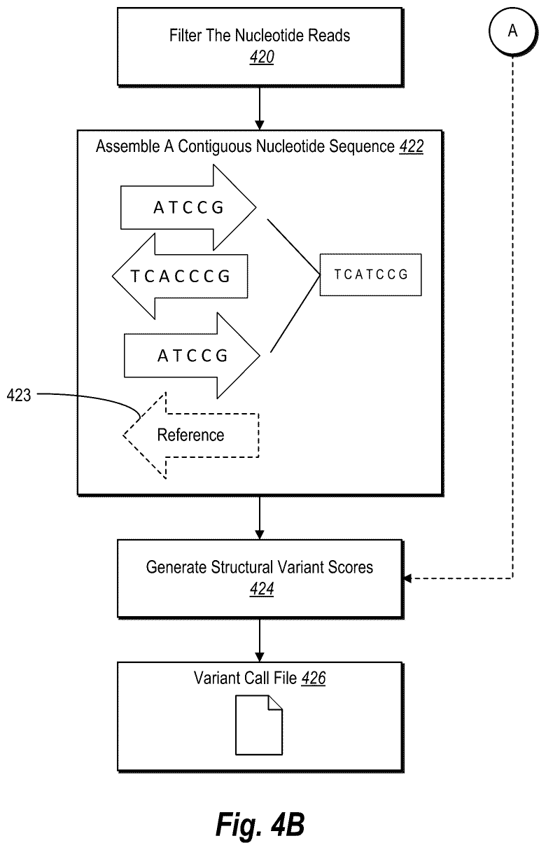

FIGS. 4A-4B illustrate the structural-variant-aware sequencing system determining candidate genomic coordinates for structural variants and generating a variant call file in accordance with one or more implementations.

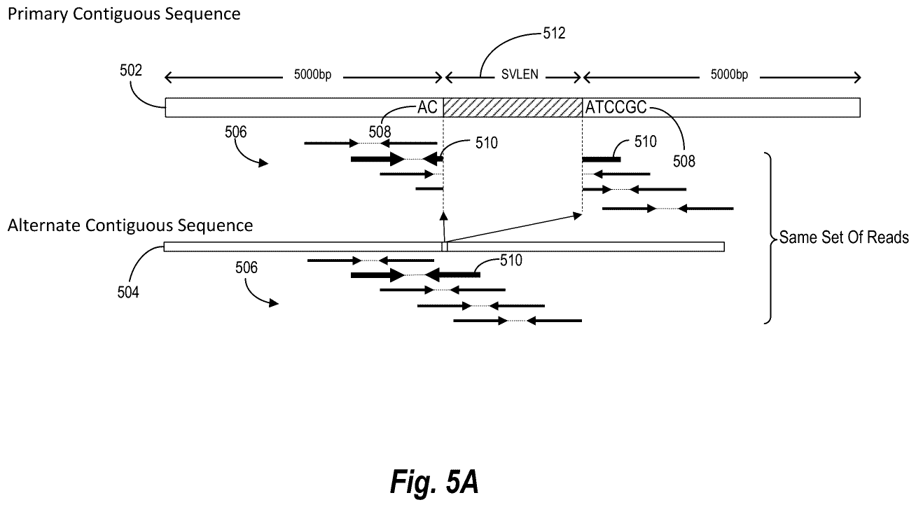

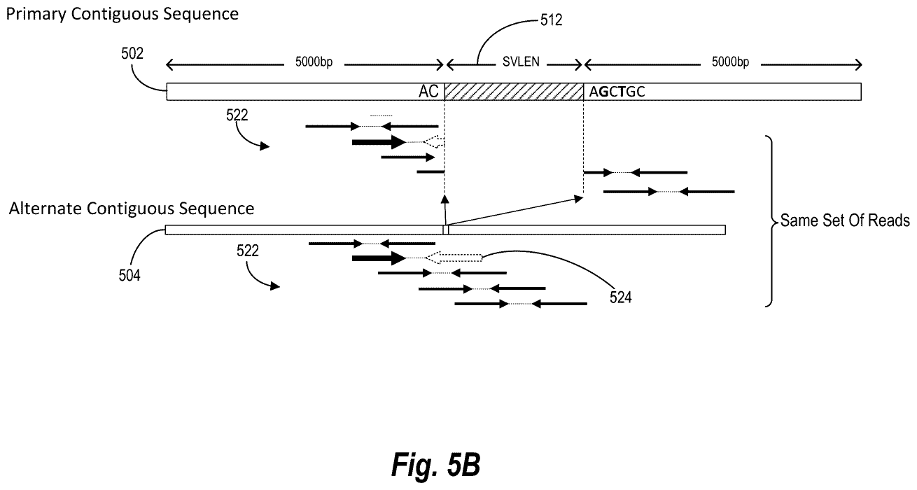

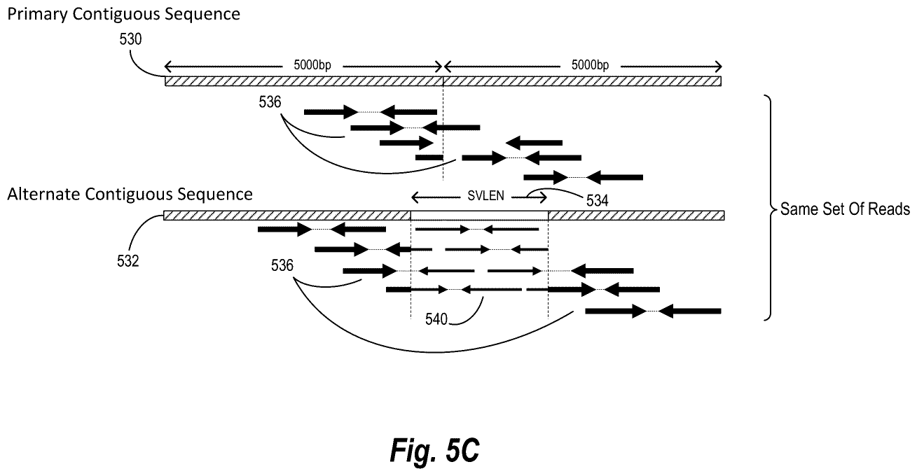

FIGS. 5A-5C illustrate the structural-variant-aware sequencing system aligning and masking one or more nucleotide reads and/or fragments of the nucleotide reads with an alternate contiguous sequence representing an insertion in accordance with one or more implementations.

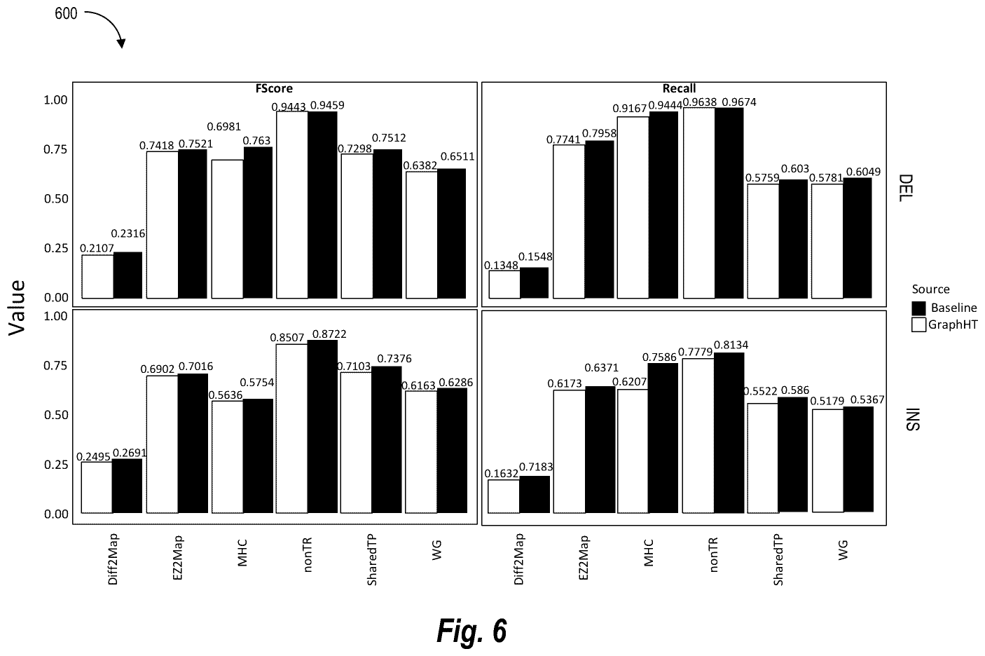

FIG. 6 illustrates improved variant detection by utilizing the disclosed structural-variant-aware sequencing system in accordance with one or more implementations.

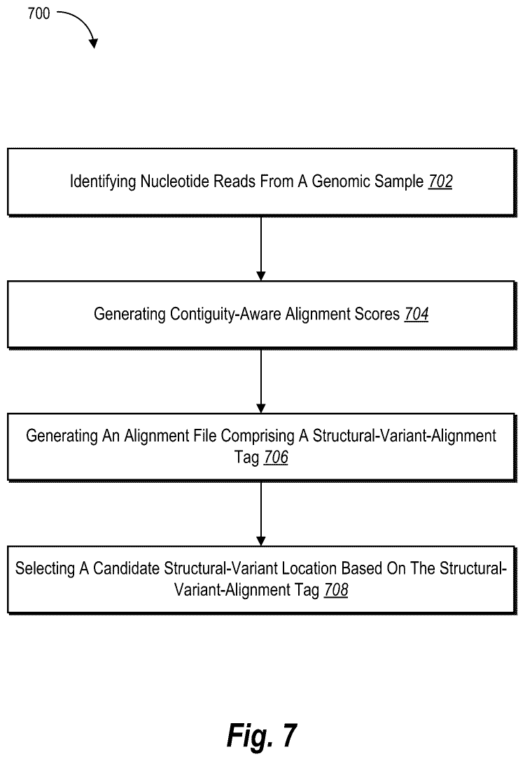

FIG. 7 illustrates a flowchart of a series of acts for generating a structural-variant-alignment tag for one or more nucleotide reads and selecting a candidate structural-variant location based on the structural-variant-alignment tag in accordance with one or more implementations.

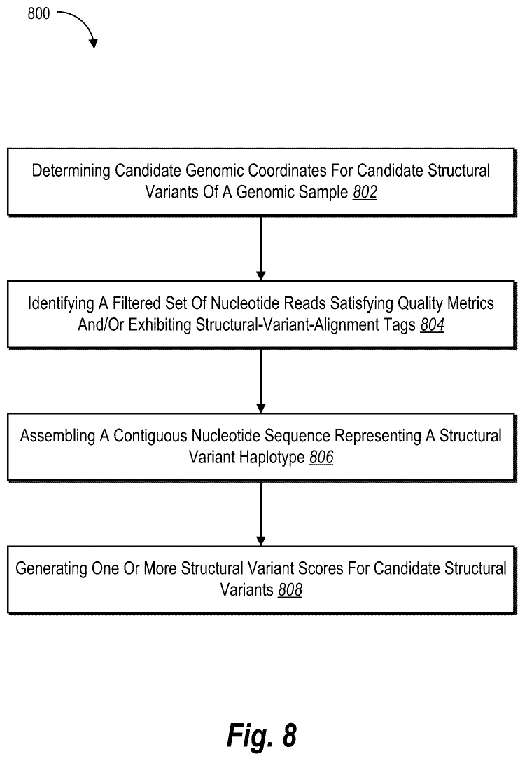

FIG. 8 illustrates a flowchart of a series of acts for generating one or more structural variant scores for candidate structural variants in accordance with one or more implementations.

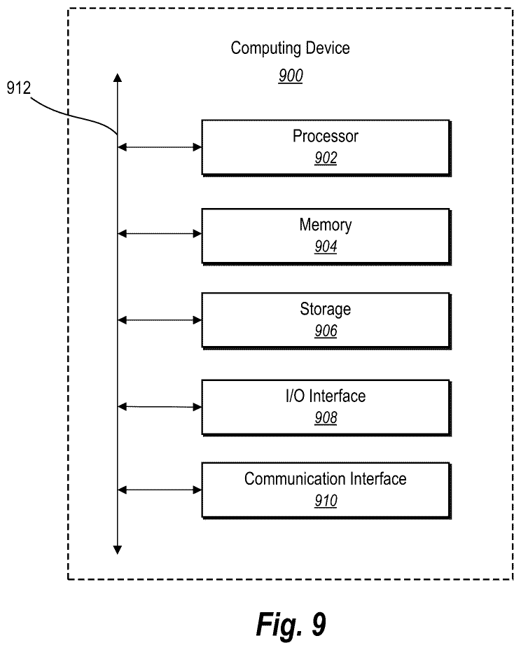

FIG. 9 illustrates a block diagram of an example computing device for implementing one or more implementations of the present disclosure.

### DETAILED DESCRIPTION

This disclosure describes one or more implementations of a structural-variant-aware sequencing system that can generate a structural-variant-alignment tag and select a candidate structural variant location based on the structural-variant-alignment tag. For example, the structural-variant-aware sequencing system identifies one or more nucleotide reads corresponding to a target genomic region of a genomic sample and analyzes candidate alignments of the one or more nucleotide read fragments with a primary contiguous sequence and/or an alternate contiguous sequence representing a structural variant haplotype. Based on the candidate alignments, the structural-variant-aware sequencing system can generate a first contiguity-aware alignment score for a candidate alignment of the nucleotide reads with the primary contiguous sequence and a second contiguity-aware alignment score for a candidate alignment of the nucleotide reads with the alternate contiguous sequence and compare the two scores. Based on the second contiguity-aware alignment score exceeding the first contiguity-aware alignment score, the structural-variant-aware sequencing system can generate a structural-variant-alignment tag indicating the one or more nucleotide reads aligns with the alternate contiguous sequence. Subsequently, the structural-variant-aware sequencing system can select the target genomic region as a candidate structural variant location based on the structural-variant-alignment tag.

As suggested above, the structural-variant-aware sequencing system can identify one or more nucleotide reads corresponding to a target genomic region of a genomic sample. For instance, the structural-variant-aware sequencing system can identify a pair of nucleotide reads corresponding to a template strand or sequence of a genomic sample. In some instances, the structural-variant-aware sequencing system receives a sequencing file with such nucleotide reads (e.g., base-call data) from a sequencing device and generates candidate alignments of the nucleotide reads from the sequencing file, including nucleotide reads corresponding any given target genomic regions.

From candidate alignments of the identified nucleotide reads, in certain implementations, the structural-variant-aware sequencing system generates contiguity-aware alignment scores. In particular, the structural-variant-aware sequencing system can generate a first contiguity-aware alignment score for a candidate alignment of one or more nucleotide reads with at least a part of the primary contiguous sequence of a structural-variation reference genome. In certain cases, the first contiguity-aware alignment score indicates the alignment accuracy of the nucleotide reads with the primary contiguous sequence. Moreover, in one or more implementations, the structural-variant-aware sequencing system generates a second contiguity-aware alignment score for a candidate alignment of one or more nucleotide reads with at least a part of an alternate contiguous sequence representing a structural variant haplotype. Akin to the first contiguity-aware alignment score, the second contiguity-aware alignment score indicates the accuracy of the alignments of the nucleotide reads with the alternate contiguous sequence. In one or more implementations, as explained further below, the contiguity-aware alignment scores are based on pair scores or fragment alignment scores.

After generating the contiguity-aware alignment scores, the structural-variant-aware sequencing system can generate an alignment file comprising data indicating predicted alignments of nucleotide reads for a genomic sample, including the identified nucleotide reads noted above. If the second contiguity-aware alignment score exceeds the first contiguity-aware alignment score, such as the second contiguity-aware alignment score represents a highest contiguity-aware alignment scores for the identified nucleotide reads, the structural-variant-aware sequencing system can generate the alignment file with a structural-variant-alignment tag indicating the candidate alignment of the identified nucleotide reads with the alternate contiguous sequence representing a structure variant haplotype. In some cases, the alignment file comprises data indicating the relative mapping position of the one or more nucleotide reads with respect to the primary contiguous sequence and/or the alternate contiguous sequence. For example, in some cases, where the alignment file comprises a structural-variant-alignment tag indicating the alignment of the one or more nucleotide reads with at least a part of the alternate contiguous sequence, the structural-variant-alignment tag can identify the location of the alignment of the nucleotide reads with the alternate contiguous sequence.

Based on the structural-variant-alignment tag, in some cases, the structural-variant-aware sequencing system selects the target genomic region as a candidate structural variant location. For example, based on the existence of the structural-variant-alignment tag, the structural-variant-aware sequencing system identifies the genomic coordinates for the target genomic region on the primary contiguous sequence as a candidate structural variant location for further filtering or later structural variant scoring.

As indicated above, in some cases, a nucleotide read aligns completely within an alternate contiguous sequence representing an insertion within the structural variant reference genome. In some such instances, the structural-variant-aware sequencing system identifies a genomic coordinate corresponding to a primary contiguous sequence at which such an alternate contiguous sequence is lifted over. The structural-variant-aware sequencing system further generates an unaligned read base indicator indicating that such an insertion-aligned nucleotide read is masked with respect to such an insertion-marker genomic coordinate and not aligned with respect to a corresponding primary contiguous sequence.

By contrast, in some instances, a nucleotide read contains a break or space (e.g., breakpoint) between a read fragment of a nucleotide read that aligns best with a primary contiguous sequence and another read fragment of the nucleotide read that aligns best with an alternate contiguous sequence representing an insertion within the structural variant reference genome. In such cases, the structural-variant-aware sequencing system can utilize the lift-over relationship between the primary contiguous sequence and alternate contiguous sequence to guide clipping (e.g., soft clipping or hard clipping) nucleobases within the read fragment aligned with the insertion-representing alternate contiguous sequence.

In addition to structural-variant-alignment tags and other alignment file information, the structural-variant-aware sequencing system introduces a new approach to calling structural variants for a genomic sample. As explained below, for example, the structural-variant-aware sequencing system can (i) determine candidate genomic coordinates for structural variants corresponding to reads exhibiting abnormal alignments or exhibiting structural-variant-alignment tags in an alignment file, (ii) identify, at candidate genomic coordinates, filtered sets of reads that satisfy quality metrics (e.g., threshold MAPQ, soft-clip criteria, concise idiosyncratic gapped alignment report (CIGAR) with I/D operations) and/or that exhibit a structural-variant-alignment tag in the alignment file; (iii) optionally assemble, from the filtered sets of reads, a contiguous sequence representing the structural variant haplotype of the genomic sample; and (iv) optionally generate, for the genomic sample, structural variant scores for a structural variant call based on an allele frequency corresponding to the structural variant haplotype and alignment of the identified nucleotide reads with the contiguous nucleotide sequence and/or a corresponding primary contiguous sequence. As described below, in some embodiments, the structural-variant-aware sequencing system generates such scores without using an allele frequency corresponding to a structural variant haplotype but rather another (e.g., default) allele frequency.

Based on a structural-variant-alignment tag and structural variant scores, in some cases, the structural-variant-aware sequencing system can generate a structural variant call for a target genomic region of a genomic sample. As mentioned above, in some embodiments, the structural-variant-aware sequencing system identifies nucleotide reads from the genomic sample. In some cases, the structural-variant-aware sequencing system further aligns the one or more nucleotide reads with at least a part of the alternate contiguous sequence representing a structural variant haplotype. Based on the aligned subset of nucleotide reads, the structural-variant-aware sequencing system can generate variant calls for the genomic sample.

As indicated above, the structural-variant-aware sequencing system provides several technical advantages relative to existing sequencing systems by improving read-alignment and base-calling accuracy. For example, the structural-variant-aware sequencing system improves the accuracy of read alignments by generating or utilizing a structural-variant-alignment tag. To illustrate, in some cases, fragments of one or more nucleotide reads span a breakpoint within the primary contiguous sequence of the structural-variant reference genome resulting in a partial alignment with the primary contiguous sequence. In such embodiments, the nucleotide read fragments can have a better alignment with the alternate contiguous sequence because they fully or partially align with an alternate contiguous sequence representing a deletion of a threshold number of base pairs, an insertion of a threshold number of base pairs, or another structural variant. In some such cases, the alternate contiguous sequence can account for a breakpoint and the structural-variant-alignment tag can indicate a more accurate alignment with the alternate contiguous sequence.

To further illustrate improved structural variant alignment, when alignments of read fragments of nucleotide reads aligns completely within an alternate contiguous sequence representing an insertion of a threshold number of base pairs, the insertion could occur at various positions on the primary contiguous sequence while still comprising the same sequence in the insertion. To resolve the ambiguity in of an insertion location, the structural-variant-aware sequencing system utilizes the liftover relationship between the alternate contiguous sequence and the primary contiguous sequence to guide the alignments to an insertion-marker genomic coordinate on the primary contiguous sequence. Thus, in certain implementations, the structural-variant-aware sequencing system can generate a clean pileup of nucleotide reads indicating evidence of the insertion by aligning the nucleotide reads to a single location on the primary contiguous sequence.

In addition to improving nucleotide read alignments, the structural-variant-aware sequencing system improves accuracy of variant calling across various genomic contexts. For example, in one or more cases, the structural-variant-alignment tag increases the pool of candidate structural variant locations. By increasing the number of candidate structural variant locations and as further illustrated by the graphs described below, the structural-variant-aware sequencing system improves the F-score and recall for insertions and deletions of a threshold number of nucleobases across various genomic regions, including difficult-to-call regions, Major Histocompatibility Complex (MHC) regions, and other regions within a reference genome.

As illustrated by the foregoing discussion, the present disclosure utilizes a variety of terms to describe features and advantages of the structural-variant-aware sequencing system. As used herein, for example, the term “variant” refers to a nucleobase or multiple nucleobases that do not align with, differ from, or vary from a corresponding nucleobase (or nucleobases) in a reference sequence or a reference genome. For example, a variant includes a SNP, an indel, or a structural variant that indicates nucleobases in a sample that differ from nucleobases in corresponding genomic coordinates of a reference sequence or a reference genome.

Relatedly, as further used herein, the term “structural variant” refers to a variation (e.g., deletion, insertion, translocation, inversion) in a structure of an organism's chromosome or a variation to the nucleotide sequences of the organism's chromosome. In some cases, a structural variant includes a variation to a threshold number of base pairs (e.g., >50 base pairs) within an organism's chromosome. Accordingly, in certain implementations, a structural variant includes an insertion or deletion exceeding a threshold number of base pairs, a duplication exceeding a threshold number of base pairs, an inversion, a translocation, or a copy number variation (CNV). While this disclosure describes some examples of 50 base pairs as a threshold number of base pairs, in some embodiments, the threshold number of base pairs for a structural variant may be different, such as 16, 25, 32, 35, 45, 100, or 1,000 base pairs.

As used herein, the term “nucleotide read” (or simply “read”) refers to an inferred sequence of one or more nucleotide bases (or nucleobase pairs) from all or part of a sample nucleotide sequence (e.g., a sample genomic sequence, complementary DNA). In particular, a nucleotide read includes a determined or predicted sequence of nucleobase calls for a nucleotide fragment (or group of monoclonal nucleotide fragments) from a sequencing library corresponding to a genomic sample. For example, in some embodiments, the call recalibration system determines a nucleotide read by generating nucleobase calls for nucleobases passed through a nanopore of a nucleotide-sample slide, determined via fluorescent tagging, or determined from a well in a flow cell. In some cases, a nucleotide read can refer to a particular type of read, such as a nucleotide read synthesized from sample library fragments that are shorter than a threshold number of nucleobases (e.g., SBS reads). In these or other cases, another type of nucleotide read can refer to (i) assembled nucleotide reads that have been assembled from shorter nucleotide reads to form a contiguous sequence satisfying a threshold number of nucleobases, (ii) circular consensus sequencing (CCS) reads satisfying the threshold number of nucleobases, or (iii) nanopore long reads satisfying the threshold number of nucleobases.

Also, as used herein, the term “genomic sample” refers to a target genome or portion of a genome undergoing an assay or sequencing. For example, a genomic sample includes one or more sequences of nucleotides isolated or extracted from a sample organism (or a copy of such an isolated or extracted sequence). In particular, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device.

Additionally, as used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). In some cases, a genomic coordinate refers to a genomic coordinate on a sex chromosome (e.g., chrX or chrY). Consequently, the call recalibration system can determine genotype probabilities and/or variant call classifications for a genotype call (e.g., a variant call) for a genomic coordinate on a sex chromosome. Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt: 16568 or SARS-CoV-2:29001). By contrast, in certain cases, a genomic coordinate refers to a position of a nucleobase within a reference genome without reference to a chromosome or source (e.g., 29727).

Relatedly, a “genomic region” refers to a range of genomic coordinates. Like genomic coordinates, in certain implementations, a genomic region may be identified by an identifier for a chromosome and a particular position or positions, such as numbered positions following the identifier for a chromosome (e.g., chr1: 1234570-1234870). In various implementations, a genomic coordinate includes a position within a reference genome (e.g., structural-variation reference genome). In some cases, a genomic coordinate is specific to a particular reference genome.

As used herein, the term “split group” refers to a group of one or more fragment alignments corresponding to a nucleotide read. In particular, a split group comprises a chain of one or more fragment alignments forming a split-alignment of one nucleotide read with respect to a reference genome. For example, a split group may comprise fragment alignments of one or more fragments of a nucleotide read. Such fragment alignments can represent alignments of read fragments from a single-end nucleotide read or a paired-end nucleotide read (e.g., a mate) from a pair of paired-end nucleotide reads. Because a split group can include a single, contiguous, fragment alignment from a single nucleotide read, in some cases, a split group includes an unbroken nucleotide read as a candidate split group. Relatedly, the term “candidate split group” refers to potential fragment alignments of one nucleotide read.

Further, the term “predicted split group” refers to a selected split group to represent an alignment of a nucleotide read. In particular, a predicted split group includes a split group having a highest split group score from among candidate split groups corresponding to a nucleotide read. In some embodiments, a predicted split group accordingly represents a prediction that the corresponding split alignment most likely represents a true alignment of the nucleotide read with a reference genome. For example, in certain circumstances described below, the predicted split group may represent a split read alignment corresponding to a true structural variant in the sequenced genomic sample.

As used herein, the term “split group score” refers to a numeric score, metric, or other quantitative measurement indicating an accuracy of fragment alignments in a split group. For instance, a split group score indicates the likelihood that a given split alignment of one or more fragment alignments of a candidate split group is correct with respect to a reference genome. For example, as explained below, a split group score may reflect a combination of fragment alignment scores, a break penalty, an overlap penalty, and, in some cases, a gap penalty for fragment alignments within a split group.

As further used herein, the term “fragment alignment” (or “read fragment alignment”) refers to a candidate local alignment of a given fragment of a nucleotide read with respect to a reference genome. For example, a fragment alignment indicates a genomic region or genomic coordinates of a reference genome with which a fragment of a read aligns.

As used herein, the term “contiguity-aware alignment score” refers to a composite numeric score, metric, or other quantitative measurement evaluating both (a) an accuracy of an alignment between a nucleotide read (or a fragment of the nucleotide read) and another nucleotide sequence from a reference genome (e.g., structural-variation reference genome) and (b) a contiguous alignment of fragments of the nucleotide read. For example, a contiguity-aware alignment score includes (i) a numeric score evaluating an accuracy of an alignment between a pair of nucleotide reads—including fragments of nucleotide reads within the pair that form candidate split groups—and primary or alternate contiguous sequences of a reference genome and (ii) a presence of breaks, gaps, or overlap between the fragments of the nucleotide reads. In some cases, a contiguity-aware alignment score further accounts for (iii) a likelihood that fragment alignments are mates (or part of mates) of a paired-end read. For instance, a contiguity-aware alignment score can include both an alignment score and alignment-score adjustments that account (e.g., penalize the alignment score) for breaks between fragment alignments of a split group, gaps between a pair of fragment alignments within the split group, or overlap between fragment alignments within the split group—as well as a penalty for fragment alignments as unlikely mates of a paired-end read. In some cases, a contiguity-aware alignment score is a pair score, as defined below, that can account for a break penalty, a gap penalty, and/or an overlap penalty.

While this disclosure describes the structural-variant-aware sequencing system determining contiguity-aware alignment scores for candidate alignments of nucleotide reads with primary contiguous sequences and/or alternate contiguous sequences representing structural variant haplotypes, in some embodiments, the structural-variant-aware sequencing system instead determines alignment scores (as defined below) for candidate alignments of nucleotide reads with such primary contiguous sequences and/or such alternate contiguous sequences. Such an alignment score need not account for breaks, gaps, and/or overlapping among read fragment alignments. For conciseness, however, this disclosure describes the structural-variant-aware sequencing system determining contiguity-aware alignment scores.

Relatedly, an “alignment score” refers to a composite numeric score, metric, or other quantitative measurement evaluating an accuracy of an alignment between a nucleotide read (or a fragment of the nucleotide read) and another nucleotide sequence from a reference genome. In particular, an alignment score includes a metric indicating a degree to which the nucleobases of a nucleotide read (or fragment of the nucleotide read) match or are similar to a primary contiguous sequence and/or an alternate contiguous sequence from a reference structural variant genome. In certain implementations, an alignment score takes the form of a Smith-Waterman score or a variation or modified version of a Smith-Waterman score for local alignments, such as various settings or configurations used by DRAGEN by Illumina, Inc. for Smith-Waterman scoring. Accordingly, the term “fragment alignment score” refers to an alignment score for a fragment alignment of a nucleotide read. In a split group comprising multiple fragment alignments, a fragment alignment score may be determined for each fragment alignment within the split group.

Relatedly, the term “pair score” refers to a numeric score, metric, or other quantitative measurement evaluating an accuracy of alignments between a candidate pair of split groups and nucleotide sequences from a reference genome. In particular, a pair score includes a metric indicating a degree to which a candidate pair of split groups is accurately aligned with a nucleotide sequence from a reference genome. More specifically, in some embodiments, a pair score indicates a likelihood that a candidate pair of split groups comprise true mates of a paired-end nucleotide read. Indeed, in some embodiments, a pair score represents a sum of split group scores for respective candidate pairs of split groups minus a pairing penalty. As noted above, a pair score is an example of a contiguity-aware alignment score.

Relatedly, the term “overlap penalty” refers to a numeric score, metric, or other quantitative measurement penalizing fragment alignments within a split group that overlap within a nucleotide read. In particular, an overlap penalty can include a metric that penalizes fragment alignments of a split group to a degree (or in proportion to) the fragment alignments exhibit overlapping nucleotide bases within a nucleotide read. For example, a 150-base-pair nucleotide read may have at least two fragment alignments. The first fragment alignment may align with the leftmost 100 base pairs to one chromosome within a reference genome (e.g., Chr1), and the second fragment alignment may align with the rightmost 100 base pairs to another chromosome (e.g., Chr2). Despite the example fragment alignments not overlapping within the reference genome, the first and second fragment alignments may nevertheless overlap by 50 base pairs within the nucleotide read. An overlap penalty can accordingly represent a metric penalizing such a 50-base-pair overlap within the nucleotide read from the foregoing example (or other example overlap of nucleotide bases).

As further used herein, the term “gap penalty” refers to a numeric score, metric, or other quantitative measurement penalizing a pair of fragment alignments based on a gap between the pair of fragment alignments within a nucleotide read. In particular, the gap penalty can include a metric that penalizes fragment alignments of a split group to a degree (or in proportion to) the size of a gap existing between the fragment alignments within a nucleotide read. For example, a 150-base-pair nucleotide read may have at least two fragment alignments. The first fragment alignment may align the leftmost 50 base pairs to a first set of genomic coordinates of a reference genome, and the second fragment alignment may align the rightmost 50 base pairs to a second set of genomic coordinates of the reference genome. In contrast to the overlap example above, the nucleotide read may include a 50 base-pair gap within the nucleotide read in between a first fragment corresponding to the first fragment alignment and a second fragment corresponding to the second fragment alignment. A gap penalty can accordingly represent a metric penalizing such a 50 base-pair gap between the first fragment alignment and the second fragment alignment within the nucleotide read.

As used herein, the term “pairing penalty” refers to a numeric score, metric, or other quantitative measurement penalizing a pair of fragment alignments that are unlikely mates of a paired-end read. In particular, the term pairing penalty refers to a metric indicating a likelihood or unlikelihood of fragment alignments being correctly paired based on a geometry of two or more fragment alignments with respect to a reference genome. For example, the pairing penalty can represent a log likelihood or, alternatively, a log P-value of an insert size between two innermost fragment alignments based on an empirical insert distribution. In some embodiments, the structural-variant-aware sequencing system does not assess or impose a pairing penalty on a pair of fragment alignments in which (i) a first nucleotide read of a paired-end read aligns with a primary contiguous sequence and (ii) a second nucleotide read of the paired-end read aligns with an alternate contiguous sequence representing a structural variant haplotype, where the alternate contiguous sequence exhibits a liftover relationship with respect to the relevant primary contiguous sequence. Similarly, the structural-variant-aware sequencing system does not assess or impose a pairing penalty on a pair of fragment alignments that align with (i) a portion of a primary contiguous sequence and (ii) some or all of an alternate contiguous sequence representing a structural variant haplotype, where the alternate contiguous sequence exhibits a liftover relationship with respect to the portion of the primary contiguous sequence. By not assessing or imposing such a pairing penalty, the structural-variant-aware sequencing system does not score such a paired-end-read alignment or a pair of fragment alignments to indicate they are unlikely mates of a paired-end read when aligned in part with both a primary contiguous sequence and such an SV alternate contiguous sequence.

As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequence determined as representative of an organism. For example, a linear human reference genome may be GRCh38 (or other versions of reference genomes) from the Genome Reference Consortium. While GRCh38 may include alternate contiguous sequences representing alternate haplotypes, GRCh38 includes alternate haplotypes with limited representation of population structural variants. Indeed, the structural variants represented in GRCh38 include only those represented by the 11 individuals whose libraries GRCh38 is constructed upon.

As disclosed herein, the term “structural-variation reference genome” refers to a reference genome that includes primary contiguous sequences (e.g., from a linear reference genome) and alternate contiguous sequences representing entire or partial structural variant haplotype sequences. For instance, in some embodiments, a structural variation reference genome includes a linear reference genome with primary contiguous sequences that has been supplemented with alternate contiguous sequences representing structural variant haplotypes and non-structural variant haplotypes. In certain cases, the structural-variation reference genome can represent structural variant haplotype sequences spanning a breakpoint. For instance, in some cases, a structural-variation reference genome represents an inversion with an alternate contiguous sequence representing either start or end sequences near (<1 kbp from) the inversion's breakpoints. In addition to alternate contiguous sequences representing structural variant haplotypes and non-structural variant haplotypes, in some embodiments, a structural variation reference genome comprises alternate nucleobases or additional alternate contiguous sequences representing alternate haplotypes, such as SNPs and/or indels below a threshold number of base pairs (e.g., <50 base pairs). In some cases, the structural-variant-aware sequencing system can represent and use the structural variation reference genome in the form of a graph hash table or other digital organization structure without nodes. For instance, a structural variation reference genome may include the Illumina DRAGEN Graph Reference Genome hg19 or later version. By contrast, in some cases, the structural variant reference genome can take the form of a true graph comprising nodes and edges. Further, in some embodiments, the structural-variation reference genome takes the form of a structural variation graph genome, as described by Generating and Implementing a Structural Variation Graph Genome, U.S. Patent Application No. 63/367,075 (filed Jun. 27, 2022), which is hereby incorporated by reference in its entirety.

As used herein, the term “primary contiguous sequence” (or simply “primary contig”) refers to a contiguous sequence representing a primary or default reference sequence from a reference genome (e.g., linear reference genome) at a particular genomic region or genomic coordinate. For instance, the primary contiguous sequence can represent a region and/or coordinate within the structural variation reference genome. To further illustrate the primary contiguous sequence can be a coordinate or region of CRGh38 from the Genome Reference Consortium.

Relatedly, the term “alternate contiguous sequence” (or simply “alt contig”) refers to a contiguous sequence representing a population haplotype added to a reference genome (e.g., linear reference genome) at a particular genomic coordinate or genomic coordinates (e.g., lifted over to the linear reference genome). In some implementations, a structural-variant reference genome can include alternate contiguous sequences mapped to genomic coordinates of a primary assembly (e.g., a full set of primary contigs) for a linear reference genome. For example, an alternate contiguous sequence may represent a population haplotype containing a structural variant with liftover to two or more genomic coordinates in the linear reference genome corresponding to two or more flanks of structural variant breakends. In certain cases, alternate contiguous sequences from GRCh38 are not mapped to the primary contiguous sequence and remain unplaced with respect to the primary contiguous sequence. In some cases, a hash table for a structural variant reference genome includes identifiers that associate alternate contiguous sequences representing structural variant haplotypes with genomic coordinates representing reference haplotypes from a primary assembly for a linear reference genome. While this disclosure repeatedly refers to alternate contiguous sequences representing structural variant haplotypes, the terms “SV alternate contiguous sequences” and “SV alt contigs” can and have been used interchangeably.

Relatedly, the term “alt-contig fragment alignment score” refers to an alignment score for an alignment between one or more read fragments with an alternate contiguous sequence. In particular, an alt-contig fragment alignment score can include an alignment score for an alignment of one or more inner read fragments and one or more outer read fragments of a nucleotide read with an alternate contiguous sequence. As explained below, an alt-contig fragment alignment score may replace or serve as a split group score under certain circumstances.

As used herein, the term “structural variant haplotype” refers to a structural variant that is present in an organism (or organisms from a population) and that is inherited from one or more ancestors as part of a grouping of nucleotide sequences. In particular, a structural variant haplotype can include a group of alleles including (or representing) one or more structural variants present in organisms of a population that tend to be inherited together by such organisms from a single parent. For instance, a structural variant haplotype can include a deletion, insertion, duplication, inversion, translocation or copy number variation (CNV). In some embodiments, a structural variant haplotype includes a deletion, and insertion, a duplication of more than fifty base pairs. In some cases, the structural variant haplotype comprises a deletion, insertion, or duplication of more than twenty-five base pairs. In some embodiments, the structural variant haplotype is a flexible range that change based on the genomic sample. Accordingly, a structural variant haplotype may include a structural variant and other variants as part of a group of alleles and may correspond to a particular gene.

As further used herein, the term “alignment file” refers to a digital file that indicates the relative alignment or mapping of nucleotide reads with nucleotide sequences of a reference structural variation genome or other reference nucleotide sequences. In particular, an alignment file can include data indicating relative mapping position of nucleotide reads and nucleotide sequences of a reference genome. In one or more implementations, the alignment file includes a structural-variant-alignment tag. In some embodiments, an alignment file includes or constitutes a Sequence Alignment/Map (SAM) file and/or a Binary Alignment Map (BAM) file.

Relatedly as used herein, the term “Sequence Alignment/Map file” (or simply “SAM file”) refers to a text format for storing sequence alignment data with a reference genome in a series of columns. In particular, the SAM file can include an alignment section with 12 fields describing various aspects of a sequence alignment. For example, the SAM file includes fields comprising QNAME, FLAG, RNAME, POS, MAPQ, CIGAR, RNEXT, PNEXT, TLEN, SEQ, QUAL, TAGS.

As used herein, the term “structural-variant-alignment tag” refers to an identifier (e.g., a TAG in a SAM or BAM file) indicating an alignment of one or more nucleotide reads to an alternate contiguous sequence representing a structural variant haplotype within a reference genome. In some embodiments, the structural-variant-alignment tag is a string tag documented in an alignment file (e.g., a SAM or BAM file). For example, the structural-variant-alignment tag can include an alternate-sequence identifier, an offset position, a strand-direction identifier, a concise idiosyncratic gapped alignment report (CIGAR), a mapping quality score, and/or an edit distance between nucleobases, as further explained below. In some embodiments, the structural-variant-alignment tag represents the alignment of one or more nucleotide reads to an alternate contiguous sequence representing a structural variant haplotype by collapsing an alignment reference into the structural-variant-alignment tag. Moreover, in some cases, the structural-variant-alignment tag provides auxiliary alignment information for a nucleotide read and or fragments of the nucleotide reads that do not have corresponding liftover coordinates on the structural-variation reference genome.

As used herein, the term “split alignment” refers to an alignment of different fragments of a nucleotide read to different regions in a reference genome. For example, a split alignment can refer to a split-read or chimeric alignment.

Relatedly, as used herein, the term “split-alignment tag” refers to an identifier (e.g., a TAG in a SAM or BAM file) indicating a split alignment of one or more nucleotide reads with another nucleotide sequence within a reference genome. In particular, the split-alignment tag indicates an alignment of different fragments of a nucleotide read to different regions on the primary contiguous sequence in the structural-variation reference genome. In some embodiments, the split-alignment tag is a string tag documented in the SAM file.

As used herein, the term “variant call file” refers to a digital file that indicates or represents one or more nucleobase calls (e.g., variant calls) compared to a reference genome along with other information about the nucleobase calls (e.g., variant calls). For example, a variant call format (VCF) file refers to a text file format that contains information about structural variants at specific genomic coordinates, including meta-information lines, a header line, and data lines where each data line contains information about a single nucleobase call (e.g., a single variant).

As used herein, the term “candidate structural-variant location” refers to a candidate or potential genomic coordinate or genomic region for a candidate structural variant. Accordingly, a candidate structural-variant location does not necessarily identify a structural variant of a genomic sample, but rather a genomic coordinate or genomic region of a candidate structural variant of the genomic sample. In some cases, an alignment file or record includes an identifier or marker indicating a genomic coordinate or genomic region exhibiting an abnormal read alignment to, accordingly, mark or identify a candidate structural-variant location. In one or more implementations, an alignment file or record includes a structural-variant-alignment tag comprising data indicating a candidate structural variant location. In some cases, the structural-variant-aware sequencing system detects structural variants by analyzing one or more candidate structural variant locations.

As used herein, the term “masking” refers to hiding, soft-clipping, or hard-clipping one or more nucleobase identifiers (e.g., A, T, C, G) within a nucleotide read (or a fragment of a nucleotide read) that do not align or match a portion of a reference genome. For example, masking can hide one or more fragments of a nucleotide read that do not align or match (e.g., to a degree of edit distance) with a portion of a primary contiguous sequence but align with an alternate contiguous sequence. In some embodiments, the structural-variant-aware sequencing system masks a portion of the nucleotide reads at a breakpoint position. In certain implementations, the structural-variant-aware sequencing system fully masks (e.g., hard or soft clips) the nucleotide reads at an insertion-marker genomic coordinate.

As used herein, the term “breakpoint” refers to a break or space between nucleotide reads and/or fragments of nucleotide reads where nucleotide reads aligns with different locations within a reference genome (e.g., structural-variation reference genome). For example, a split alignment contains a breakpoint because the fragments of the nucleotide read have the highest scoring alignments with the structural-variation reference genome when they align to different locations that have a break or breakpoint between them.

As used herein, the term “liftover relationship” refers to a mapping relationship between an alternate contiguous sequence representing a variant haplotype and a genomic coordinate or genomic region of a primary contiguous sequence within a reference genome. In particular, a liftover relationship maps the alternate contiguous sequence representing a structural variant haplotype or other variant haplotype to genomic coordinates of the primary contiguous sequence in the structural-variation reference genome. For example, the liftover relationship between the alternate contiguous sequence and corresponding primary contiguous sequence can guide read alignments to a corresponding insertion-marker genomic coordinate. In some embodiments, the structural-variant-aware sequencing system generates a Sequence Alignment/Map (Sam) liftover file dictating the liftover relationship between the alternate contiguous sequence and primary contiguous sequence. For instance, the liftover file can provide evidence of masked nucleotide reads aligned at the insertion-marker genomic coordinate.

Relatedly, as used herein, the term “insertion-marker genomic coordinate” refers to a genomic coordinate of the primary contiguous sequence associated with an alternate contiguous sequence representing an insertion of a threshold number of base pairs based on the liftover relationship. For example, the insertion-marker genomic coordinate, in an alignment file, with respect to which a nucleotide read (or a fragment of a nucleotide read) aligns completely within an insertion and is masked. Accordingly, an insertion-marker genomic coordinate identifies a genomic coordinate for a primary contiguous sequence within a reference genome from which nucleobases within a nucleotide read are masked. Accordingly, an insertion-marker genomic coordinate is itself a type of genomic coordinate, as described above.

As used herein, the term “structural variant score” refers to a score indicating a likelihood or a probability of a genomic sample comprising a structural variant given the observed nucleotide reads. In some embodiments, a structural variant score may include a genotype probability that a genomic sample comprises a candidate structural variant at a genomic coordinate or a genomic region. As explained further below, in some embodiments, a structural variant score includes a posterior probability that a genomic sample exhibits a candidate structural variant in whole or in part of a genotype at one or more genomic coordinates. In certain cases, the structural variant scores are based on an allele frequency corresponding to the structural variant haplotype. For instance, the structural variant score can be a posterior probability of a genotype given the nucleotide reads (or nucleotide read fragments) for a certain allele exhibited by a genomic sample.

As further used herein, the term “nucleobase call” (or simply “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or another base-call-output file-based on nucleotide reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, a thymine (T) call, or an uracil (U) call.

As used herein, the term “abnormal alignments” refers to nucleotide read alignments or fragment alignments that deviate from a mean or standard relative to other nucleotide read alignments or fragment alignments from a same genomic sample. In particular, abnormal alignments include nucleotide read alignments or fragment alignments exhibiting an anomalous masking of nucleobases or indicating an insert size that satisfies or exceeds a threshold size. In some cases, abnormal alignments include a cluster of nucleotide read alignments with masked fragments or nucleobases that satisfy or exceed a threshold number of nucleobases (e.g., 15, 20, 35 nucleobases). In one or more implementations, abnormal alignments include a cluster of pairs of read fragment alignments exceeding or falling below a threshold insert size.

As used herein, an “insert” refers to sample genomic sequence (e.g., a gDNA fragment) that is extracted from a genomic sample and used as a template for a cluster of oligonucleotides. Accordingly, an insert includes the template for a cluster from which nucleotide reads (e.g., paired-end reads) are determined during a sequencing run. In some cases, the insert includes or excludes adapter sequences attached to the ends of the sample genomic sequence. To illustrate, in some cases, an insert includes priming and indexing sequences on either end of the sequence, while in other cases an insert excludes priming and indexing sequences.

As further used herein, the term “threshold insert size” refers to a threshold indicating an abnormal size or length of an insert or genomic fragment corresponding to a nucleotide read. Such an insert or genomic fragment represents a genomic sequence within a sample library fragment corresponding to a particular genomic sample. In some embodiments, the threshold insert size is dynamic and changes based on the number of genome samples. To illustrate, the threshold insert size can be based on the mean value of an insert corresponding to nucleotide reads (e.g., paired-end reads) and the standard deviation from the mean.

As used herein, for example, the term “configurable processor” refers to a circuit or chip that can be configured or customized to perform a specific application. For instance, a configurable processor includes an integrated circuit chip that is designed to be configured or customized on site by an end user's computing device to perform a specific application. Configurable processors include, but are not limited to, an ASIC, ASSP, a coarse-grained reconfigurable array (CGRA), or FPGA. By contrast, configurable processors do not include a CPU or GPU. In some embodiments, the structural-variant-aware sequencing system uses a configurable processor (e.g., FPGA) or a processor (e.g., CPU) to perform the various embodiments described herein.

As used herein, the term “base-call-quality metric” refers to a specific score or other measurement indicating an accuracy of a nucleobase call. In particular, a base-call-quality metric comprises a value indicating a likelihood that one or more predicted nucleobase calls for a genomic coordinate contain errors. For example, in certain implementations, a base-call-quality metric can comprise a Q score (e.g., a PHil's Read EDitor (PHRED) quality score) predicting the error probability of any given nucleobase call. To illustrate, a quality score (or Q score) may indicate that a probability of an incorrect nucleobase call at a genomic coordinate is equal to 1 in 100 for a Q20 score, 1 in 1,000 for a Q30 score, 1 in 10,000 for a Q40 score, etc.

Also, as used herein, the term “mapping quality score” refers to a metric or other measurement quantifying a quality or certainty of an alignment of nucleotide reads or other sample nucleotide sequences with a reference genome. In particular, a mapping quality score includes MAPQ scores for nucleotide reads at genomic coordinates, where a MAPQ score represents −10 log 10 Pr{mapping position is wrong}, rounded to the nearest integer.

As used herein, the term “imputation model” refers to a statistical method used to estimate a likelihood that a sample comprises a particular nucleobase or haplotype based on a dataset corresponding to the sample. In particular, an imputation model applies algorithms to infer the presence of one or more haplotypes in a genomic sample based on (i) nucleotide reads of the genomic sample, such as reads aligned with a reference genome, and (ii) patterns and relationships within the nucleotide reads. For instance, in some cases, an imputation model utilizes an algorithm, such as Hidden Markov Model (HMM) based imputation, regression-based imputation, or machine learning algorithms, to infer a likelihood of a presence of a particular nucleobase or a particular haplotype within a genomic sample based on read alignments (e.g., based on flanking nucleotide reads) that may not directly indicate the presence of the particular nucleobase or the haplotype, thereby enabling more accurate structural variant calling.

As used herein, the term “flanking nucleotide read” refers to a nucleotide read that aligns to one or more genomic regions adjacent to a target or specific genomic coordinate or region of interest. In particular, a flanking nucleotide read of a genomic sample aligns with (or covers) an adjacent genomic region within a threshold number of nucleobases (e.g., 50; 100; 150; 300; 500; 1,000; or 3,0000 nucleobases) of a target genomic region (e.g., a gene, a regulatory element, or a variant locus) of a primary contiguous sequence of a reference genome or of an alternate contiguous sequence. For instance, a flanking nucleotide read can align with nucleobases of a primary contiguous sequence or an alternate contiguous sequence immediately upstream or downstream, or within a threshold number of nucleobases upstream or downstream, of a structural variant, SNV, an indel, or a breakpoint.

As used herein, the term “candidate-likelihood threshold” refers to a likelihood or a probability used to determine whether a haplotype is present in a genomic sample. In particular, a candidate-likelihood threshold includes a cutoff value for distinguishing structural variant haplotypes with corresponding alternate contiguous sequences to be considered at a genomic coordinate or genomic region for structural variant calling of a genomic sample.

As used herein, the term “reference-guide read” refers to a nucleobase sequence that serves as a backbone or template sequence for a reference-guided assembler tool in assembling a contiguous nucleotide sequence. Specifically, a reference-guide read provides a template sequence at a specific genomic coordinate from which to fill gaps and/or to which other nucleotide reads are aligned and assembled. For instance, a reference-guide read can be a primary contiguous sequence or an alternate contiguous sequence used to guide the assembly of shorter, overlapping nucleotide reads.

The following paragraphs describe the structural-variant-aware sequencing system with respect to illustrative figures that portray example embodiments and implementations. For example, FIG. 1 illustrates a schematic diagram of a computing system 100 in which a structural-variant-aware sequencing system 106 operates in accordance with one or more embodiments. As illustrated, the computing system 100 includes a sequencing device 102 connected to a local device 108 (e.g., a local server device), one or more server device(s) 110, and a client device 114. As shown in FIG. 1, the sequencing device 102, the local device 108, the server device(s) 110, and the client device 114 can communicate with each other via a network 118. The network 118 comprises any suitable network over which computing devices can communicate. Example networks are discussed in additional detail below with respect to FIG. 9. While FIG. 1 shows an embodiment of the structural-variant-aware sequencing system 106, this disclosure describes alternative embodiments and configurations below.

As indicated by FIG. 1, the sequencing device 102 comprises a computing device and a sequencing device system 104 for sequencing a genomic sample or other nucleic-acid polymer. In some embodiments, by executing the sequencing device system 104 using a processor, the sequencing device 102 analyzes nucleotide fragments or oligonucleotides extracted from genomic samples to generate nucleotide reads or other data utilizing computer implemented methods and systems either directly or indirectly on the sequencing device 102. More particularly, the sequencing device 102 receives nucleotide-sample slides (e.g., flow cells) comprising nucleotide fragments (e.g., genomic DNA fragments) extracted from samples and further copies and determines the nucleobase sequence of such extracted nucleotide fragments.

In one or more embodiments, the sequencing device 102 utilizes SBS to sequence nucleotide fragments into nucleotide reads and determine nucleobase calls for the nucleotide reads. In addition or in the alternative to communicating across the network 118, in some embodiments, the sequencing device 102 bypasses the network 118 and communicates directly with the local device 108 or the client device 114. By executing the sequencing device system 104, the sequencing device 102 can further store the nucleobase calls as part of base-call data that is formatted as a binary base call (BCL) file and send the BCL file to the local device 108 and/or the server device(s) 110.

As further indicated by FIG. 1, the local device 108 is located at or near a same physical location of the sequencing device 102. Indeed, in some embodiments, the local device 108 and the sequencing device 102 are integrated into a same computing device. The local device 108 may run the structural-variant-aware sequencing system 106 to generate, receive, analyze, store, and transmit digital data, such as by receiving base-call data or determining variant calls based on analyzing such base-call data. As indicated by the dashed lines encompassing the structural-variant-aware sequencing system 106, the structural-variant-aware sequencing system 106 can operate/exist on the sequencing device 102, the local device 108, and/or the client device 114. As shown in FIG. 1, the sequencing device 102 may send (and the local device 108 may receive) base-call data generated during a sequencing run of the sequencing device 102. By executing software in the form of the structural-variant-aware sequencing system 106, the local device 108 may align nucleotide reads with a structural variation reference genome 112 and determine genetic variants based on the aligned nucleotide reads. In one or more cases, the structural variation reference genome 112 can reside within the structural-variant-aware sequencing system 106 on the sequencing device 102, the local device 108 and/or the client device 114. The local device 108 may also communicate with the client device 114. In particular, the local device 108 can send data to the client device 114, including a variant call format (VCF) file, alignment file, liftover file, or other information indicating variant or reference calls, sequencing metrics, error data, or other metrics.

As further indicated by FIG. 1, the server device(s) 110 are located remotely from the local device 108 and the sequencing device 102. Similar to the local device 108, in some embodiments, the server device(s) 110 include a version of the structural-variant-aware sequencing system 106. Accordingly, the server device(s) 110 may generate, receive, analyze, store, and transmit digital data, such as by receiving base-call data or determining variant calls based on analyzing such base-call data. As indicated above, the sequencing device 102 may send (and the server device(s) 110 may receive) base-call data from the sequencing device 102. The server device(s) 110 may also communicate with the client device 114. In particular, the server device(s) 110 can send data to the client device 114, including VCFs, alignment files, or other sequencing related information.

In some embodiments, the server device(s) 110 comprise a distributed collection of servers where the server device(s) 110 include a number of server devices distributed across the network 118 and located in the same or different physical locations. Further, the server device(s) 110 can comprise a content server, an application server, a communication server, a web-hosting server, or another type of server.

As indicated above, as part of the server device(s) 110 or the local device 108, the structural-variant-aware sequencing system 106 can select a target genomic region as a candidate structural variant location for variant calling. For instance, the structural-variant-aware sequencing system 106 can identify nucleotide reads corresponding to a target genomic region of a genomic sample. From the identified nucleotide reads, the structural-variant-aware sequencing system 106 can generate a first contiguity-aware alignment score for a candidate alignment of the identified nucleotide reads with at least a part of a primary contiguous sequence of a structural-variation reference genome and a second contiguity-aware alignment score for an alignment of the identified nucleotide reads with at least a part of an alternate contiguous sequence representing a structural variant haplotype within the structural-variation reference genome. Based on the second contiguity-aware alignment score exceeding the first contiguity-aware alignment score, the structural-variant-aware sequencing system 106 generates an alignment file comprising a structural-variant-alignment tag indicating the alignment of the identified nucleotides reads with at least a part of the alternate contiguous sequence. Based on the structural-variant-alignment tag, the structural-variant-aware sequencing system 106 can select the target genomic region as a candidate structural-variant location. Moreover, based on the candidate alignment of the nucleotide reads with a part of the alternate contiguous sequence and structural variant scores, in some cases, the structural-variant-aware sequencing system 106 can generate a structural variant call for the genomic sample.

As further illustrated and indicated in FIG. 1, by executing a sequencing application 116, the client device 114 can generate, store, receive, and/or send digital data. In particular, the client device 114 can receive sequencing data from the local device 108 or receive call files (e.g., BCL) and sequencing metrics from the sequencing device 102. Furthermore, the client device 114 may communicate with the local device 108 or the server device(s) 110 to receive an alignment file, a liftover file, a VCF comprising nucleobase calls and/or other metrics, such as a base-call-quality metrics or pass-filter metrics. The client device 114 can accordingly present or display information pertaining to structural variant calls or other nucleobase calls within a graphical user interface of the sequencing application 116 to a user associated with the client device 114. For example, the client device 114 can present structural variant calls, call files, and/or sequencing metrics for a sequenced genomic sample within a graphical user interface of the sequencing application 116.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 9.

As further illustrated in FIG. 1, the client device 114 includes the sequencing application 116. The sequencing application 116 may be a web application or a native application stored and executed on the client device 114 (e.g., a mobile application, desktop application). The sequencing application 116 can include instructions that (when executed) cause the client device 114 to receive data from the structural-variant-aware sequencing system 106 and present, for display at the client device 114, base-call data, data from a VCF or data from an alignment file.

As further illustrated in FIG. 1, a version of the structural-variant-aware sequencing system 106 may be located and implemented (e.g., entirely or in part) on the client device 114 or the sequencing device 102. In yet other embodiments, the structural-variant-aware sequencing system 106 is implemented by one or more other components of the computing system 100, such as the local device 108. In particular, the structural-variant-aware sequencing system 106 can be implemented in a variety of different ways across the sequencing device 102, the local device 108, the server device(s) 110, and the client device 114. For example, the structural-variant-aware sequencing system 106 can be downloaded from the server device(s) 110 to the structural-variant-aware sequencing system 106 and/or the local device 108 where all or part of the functionality of the structural-variant-aware sequencing system 106 is performed at each respective device within the computing system 100.

As indicated above, the structural-variant-aware sequencing system 106 can generate a structural-variant-alignment tag in an alignment file to guide selecting a candidate structural variant location and/or variant calling for a genomic sample. FIG. 2 depicts an overview of the structural-variant-aware sequencing system 106 generating a structural-variant-alignment tag based on contiguity-aware alignment scores and selecting the candidate structural variant location based on the structural-variant-alignment tag in accordance with one or more embodiments. As illustrated in FIG. 2, the structural-variant-aware sequencing system 106 performs a series of acts 200 including an act 202 of identifying one or more nucleotide reads. The structural-variant-aware sequencing system 106 further performs an act 204 of generating contiguity-aware alignment scores for candidate alignments of nucleotide reads with a primary contiguous sequence representing a reference haplotype and an alternate contiguous sequence representing a structural variant haplotype. The structural-variant-aware sequencing system 106 also performs an act 206 of generating an alignment file comprising a structural-variant-alignment tag and an act 208 of selecting a candidate structural variant location for a genomic sample.

As shown in FIG. 2, the structural-variant-aware sequencing system 106 performs the act 202 of identifying one or more nucleotide reads. In particular, the structural-variant-aware sequencing system 106 identifies or receives one or more nucleotide reads corresponding to a genomic region of a genomic sample. For example, the structural-variant-aware sequencing system 106 may identify nucleotide reads corresponding to a template or genomic DNA fragment sequence of a genomic sample. In some cases, the sequence of the genomic sample comprises an original contiguous DNA or RNA fragment sequence that has been sequenced and resulted in either single-end or paired-end methods. In some cases, for instance, the structural-variant-aware sequencing system 106 receives base-call data (e.g., BCL file or FASTQ file) from a sequencing device. In some such cases, the base-call data takes the form of a base-call-data file that organizes single-end reads or paired-end reads according to index sequences attached to oligonucleotides extracted from a genomic sample. In the paired-end method, a first read (e.g., R1) is sequenced from one end of the template toward the middle and a second read (e.g., R2) is sequenced from the other end. FIG. 2 illustrates two paired-end reads R1 and R2 oriented toward each other. As illustrated, there is a gap between R1 and R2, however, overlap between R1 and R2 is also possible. R1 and R2 may be described as paired-end mates.

As further illustrated in FIG. 2, the structural-variant-aware sequencing system 106 performs the act 204 of generating contiguity-aware alignment scores. In particular the structural-variant-aware sequencing system 106 generates a first contiguity-aware alignment score for a candidate alignment of one or more nucleotide reads with at least a part of a primary contiguous sequence of a structural-variation reference genome. For example, the structural-variant-aware sequencing system 106 can determine a score indicating an accuracy with which the nucleobase data represented by one or more nucleotide reads matches and aligns in whole or in part with the primary contiguous sequence from the structural-variation reference genome. As indicated above, the first contiguity-aware alignment score can account for a contiguous alignment of fragments of the identified nucleotide reads and take the form of a pair score. Additionally, the structural-variant-aware sequencing system 106 can generate a second contiguity-aware alignment score for a candidate alignment of one or more nucleotide reads with at least a part or whole of an alternate contiguous sequence representing a structural variant haplotype.

While this disclosure describes and depicts the structural-variant-aware sequencing system 106 in FIG. 2 (and elsewhere) determining contiguity-aware alignment scores for candidate alignments of nucleotide reads with primary contiguous sequences and/or alternate contiguous sequences representing structural variant haplotypes, in some embodiments, the structural-variant-aware sequencing system 106 instead determines alignment scores for candidate alignments of nucleotide reads with such primary contiguous sequences and/or such alternate contiguous sequences.

In some cases, the structural-variant-aware sequencing system 106 determines that the second contiguity-aware alignment score exceeds the first contiguity-aware alignment score and/or other contiguity-aware alignment scores for other alternative alignments of the identified nucleotide reads with primary contiguous sequences or alternate contiguous sequences. For instance, the structural-variant-aware sequencing system 106 determines that the second contiguity-aware alignment score exhibits a highest contiguity-aware alignment scores among candidate alignments of the identified nucleotide reads (e.g., different candidate alignment for R1 and R2) corresponding to the target genomic region.

As further illustrated in FIG. 2, the structural-variant-aware sequencing system 106 performs the act 206 of generating an alignment file. For instance, the alignment file includes data about selected alignments of nucleotide reads with the structural-variation reference genome. Based on the second contiguity-aware alignment score exceeding the first contiguity-aware alignment score—and/or all other contiguity-aware alignment scores for candidate alignments of the identified nucleotide reads—the structural-variant-aware sequencing system 106 generates the alignment file including a structural-variant-alignment tag indicating the candidate alignment of the identified nucleotide reads with the alternate contiguous sequence representing a structural variant haplotype. As explained further below, the structural-variant-alignment tag can include data specifying a location of the identified nucleotide reads' structural-variant candidate alignment and other information relating the identified nucleotide reads to the structural variation reference genome. This disclosure describes an example of an alignment file and a structural-variant-alignment tag below with respect to FIG. 3.

After generating the alignment file, as further shown in FIG. 2, the structural-variant-aware sequencing system 106 performs the act 208 of selecting a candidate structural variant location. In particular, the structural-variant-aware sequencing system 106 selects the structural variant location for a target genomic region of a genomic sample based on the structural-variant-alignment tag. For example, the structural-variant-alignment tag indicates the location of a candidate structural variant. In addition to genomic regions or genomic coordinates corresponding to a structural-variant-alignment tag, in one or more embodiments, the structural-variant-aware sequencing system 106 further selects one or more other candidate structural variant locations based on nucleotide reads exhibiting abnormal alignments. This disclosure describes examples of such candidate structural variant locations below with respect to FIGS. 4A and 4B.

As mentioned previously, in some embodiments, the structural-variant-aware sequencing system 106 generates an alignment file. In particular, the structural-variant-aware sequencing system 106 can generate the alignment file in a Sequence Alignment/Map (SAM) file format. In some implementations the SAM file includes a structural-variant-alignment tag. FIG. 3 illustrates an example of a structural-variant-alignment tag within an alignment file indicating an alignment of one or more identified nucleotide reads with at least a part of the alternate contiguous sequence in accordance with one or more implementations. In particular, FIG. 3 illustrates the SAM file 300 with the structural-variant-alignment tag 302.

As mentioned above, in some cases, one or more nucleotide reads exhibit a higher contiguity-aware alignment score for a candidate alignment with an alternate contiguous sequence representing a structural variant haplotype of the structural-variation reference genome than other contiguity-aware alignment scores for alignments with a primary contiguous sequence of the structural-variation reference genome. In such cases, the structural-variant-aware sequencing system 106 can document and describe in an alignment file a relatively better alignment for identified nucleotide reads (e.g., pair mates) with an alternate contiguous sequence by augmenting the alignment file to indicate one or more supplementary alignments with a structural variant.

As indicated above, the structural-variation reference genome (e.g., linear reference genome) includes additional alternate contiguous sequences. In some embodiments, the structural-variant-aware sequencing system 106 produces supplementary alignments by utilizing a graph hash table. In one or more embodiments, the structural-variant-aware sequencing system 106 collapses supplementary alignments of nucleotide reads exhibiting their best contiguity-aware alignment scores with structural-variant-representing alternate contiguous sequences and represents such alignments with structural-variant-alignment tags (e.g., structural-variant-alignment tag 302) in the SAM file 300.

As shown in FIG. 3 and mentioned above, the SAM file 300 includes information about the alignment of the nucleotide reads. In one or more cases, the SAM file 300 has certain specifications. For example, the SAM file 300 includes an optional header section 306 and an alignment section 308. The alignment section includes a QNAME field 310 (e.g., field for query name) used to identify a read within the SAM file 300. For example, in some embodiments, mated paired-end reads have the same QNAME in the QNAME field 310. As shown in FIG. 3, the alignment section includes a FLAG field 312. In some embodiments, a FLAG in the FLAG field 312 is a combination of bitwise FLAGs providing read alignment information (e.g., read paired, read unmapped, supplementary alignment, etc.). Additionally, an RNAME 314 (e.g., field for a reference sequence name) identifies a given primary or alternate contiguous sequence within the structural-variation reference genome aligned with a given nucleotide read. As further illustrated in FIG. 3, the SAM file 300 includes a POS field 316 (e.g., a field for 1-based position). In one or more embodiments, a POS value in the POS field 316 indicates the leftmost position of mapping the nucleotide read to the structural-variation reference genome.

As shown in FIG. 3, the SAM file 300 includes a MAPQ field 318 (e.g., a field for mapping quality). In particular, a MAPQ value in the MAPQ field 318 comprises a PHRED-scaled mapping quality score indicating the likelihood of correctly or incorrectly mapping the nucleotide read to a contiguous sequence of the structural-variation reference genome. In some cases, the SAM file 300 includes a CIGAR field 320 (e.g., for a concise idiosyncratic gapped alignment report string) giving an alignment summary. For example, a CIGAR in the CIGAR field 320 indicates a match, deletion, or insertion that (if performed) would match a sequence of a nucleotide read with a primary contiguous sequence.

As further illustrated in FIG. 3, the SAM file 300 includes an RNEXT field 322 (e.g., reference name for mate). In particular, an RNEXT value in the RNEXT field 322 indicates the reference sequence name of the primary alignment of the next read. In some cases, the SAM file 300 includes a PNEXT field 324 (e.g., field for a position of Mate) describing the position of the primary alignment of next read. In one or more cases, a TLEN value (e.g., template length) in a TLEN field 326 indicates the length of the template sequence to which the nucleotide reads map. In some embodiments, a plus sign indicates the read is the leftmost read. Relatedly, a minus sign indicates the read is the rightmost read.

As further indicated in FIG. 3, the SAM file 300 includes a SEQ field 328 (e.g., field for a read sequence) displaying the read sequence. Additionally, as indicated in FIG. 3, the SAM file 300 includes a QUAL field 329 (e.g., field for read quality) indicating the accuracy of each base read in the SEQ. In some embodiments, QUAL is based on the PHRED-scaled base-call-quality metric that measures base call error. Finally, FIG. 3 illustrates TAGs within the SAM file 300. In particular, a TAG is an optional field for storing and/or providing additional information about the read and/or alignment.

As just mentioned, the SAM file 300 can include one or more TAGS for providing additional information about the nucleotide reads and/or alignments. In certain cases, the SAM file 300 includes predefined TAGs for storing information about the nucleotide read and/or alignment. For example, a predefined TAG can store previous settings for various fields if additional processing updated a certain field.

Additionally, in one or more embodiments, TAGs are defined by the structural-variant-aware sequencing system 106 and store specified information about the nucleotide read and/or alignment. As shown in FIG. 3, the SAM file 300 includes a structural-variant-alignment tag 302. In some embodiments, the structural-variant-alignment tag 302 indicates where the nucleotide reads align on the alternate contiguous sequence representing the structural variant haplotype. Additionally, as indicated above, the structural-variant-alignment tag 302 indicates a selected alignment of a given nucleotide read with an alternate contiguous sequence representing a structural variant. As indicated above, the structural-variant-aware sequencing system 106 selects the candidate alignment with such an alternate contiguous sequence because the contiguity-aware alignment score for the selected candidate alignment is better than the other contiguity-aware alignment scores for alignments with one or more primary contiguous sequences or one or more other alternate contiguous sequences.

As further shown in FIG. 3, in some embodiments, the structural-variant-alignment tag 302 conforms to the format of the SAM file 300. In particular the structural-variant-alignment tag 302 takes on the TAG: TYPE: VALUE format. For example, TAG comprises a character TAG key. As shown, a TAG key 330 for the structural-variant-alignment tag 302 comprises sv. While FIG. 3 depicts the TAG key 330 with two lowercase characters for “sv,” in alternative embodiments, the TAG key 330 can comprise alternative lowercase characters (e.g., “ga” for graph alignment), two uppercase characters (e.g., “GA” for graph alignment), or any combination of lowercase and uppercase characters. Note, however, that the current SAM specifications reserve TAGs with uppercase characters for certain circumstances (e.g., TAGs relevant to primary contiguous sequences) that would not be applicable to a structural-variant-alignment tag.

In one or more embodiments, a TYPE 332 indicates the type of VALUE stored in the TAG. For example, as shown in FIG. 3, the “Z” character indicates that the structural-variant-alignment tag 302 is a string tag. In other embodiments, the structural-variant-alignment tag 302 can be a character tag, signed 32-bit integer tag, single-precision float, or hex string tag. In certain implementations the VALUE includes additional information about the nucleotide read and/or alignment. For example, as further illustrated in FIG. 3, the VALUE of the structural-variant-alignment tag 302 includes six comma separated fields comprising an alternate-sequence identifier 334, an offset position 336 of one or more nucleotide reads, a strand-direction identifier 338, a CIGAR 340, a mapping quality score 342, and an edit distance 344.

As mentioned above, the structural-variant-alignment tag 302 includes the alternate-sequence identifier 334 (e.g., alt_chr1_14255) identifying a particular alternate contiguous sequence representing a structural variant haplotype that is not part of a linear reference genome or part of an input reference file (e.g., FASTA file). Accordingly, such an SV alternate contiguous sequence as part of a structural variation reference genome provides more flexibility in terms of which structural variant haplotypes to include and does not change the coordinates or primary contiguous sequences of a linear reference genome that are part of an input reference file. Consequently, neither the structural-variant-alignment tag 302 nor the corresponding SV alternate contiguous sequence change the existing primary contiguous sequences or existing alternate contiguous sequences in GRCh38 or other standard versions of a human reference genome. By identifying the particular SV alternate contiguous sequence that is not part of a linear reference genome or an input reference file, the structural-variant-alignment tag 302 further provides a reference sequence (unlike existing tags) for a structural variant that facilitates identifying a candidate structural-variant location and structural variant scoring for candidate structural variants as part of a genomic sample's genotype.

As further indicated in FIG. 3, the structural-variant-alignment tag 302 comprises the offset position of the one or more nucleotide reads. In particular, the offset position indicates the position of the nucleotide reads with respect to the alternate contiguous sequence within the structural-variation reference genome. For example, the offset position can indicate the start position (or, in some cases, the end position) of the nucleotide read alignment with respect to the alternate contiguous sequence.

As FIG. 3 further shows, the structural-variant-alignment tag 302 includes the strand direction identifier. In particular, the strand-direction identifier indicates a forward or reverse strand of an identified nucleotide read with respect to the alternate contiguous sequence. The strand-direction identifier accordingly provides orientation information to relate the identified nucleotide read with respect to the particular alternate contiguous sequence.

As mentioned above, the structural-variant-alignment tag 302 further includes the CIGAR. In particular, the CIGAR indicates the alignment of an identified nucleotide read with the particular alternate contiguous sequence. In particular, unlike existing CIGAR's that rely on a primary contiguous sequence, the CIGAR in the structural-variant-alignment tag 302 encodes the nucleobases that match, need to be inserted or deleted, or include gaps or substitutes with respect to the particular alternate contiguous sequence identified by the alternate-sequence identifier.

As further indicated in FIG. 3, the structural-variant-alignment tag 302 includes a mapping quality score for a mapping of the identified nucleotide read to the alternate contiguous sequence identified by the alternate-sequence identifier. For example, in some embodiments, the mapping quality score is a PHRED-scaled score indicating the likelihood of incorrectly mapping the nucleotide reads to the alternate contiguous sequence. Additionally, as shown in FIG. 3, the structural-variant-alignment tag 302 includes the edit distance between the nucleobases of the nucleotide reads and the alternate contiguous sequence. For example, the edit distance can include sum of insertions, deletions, and/or substitutions between the nucleotide reads and the alternate contiguous sequence.

In contrast to the structural-variant-alignment tag 302, as further illustrated in FIG. 3, the SAM file 300 includes a split-alignment tag 304. As mentioned above, the split-alignment tag 304 indicates an alignment of different fragments of a nucleotide read to different regions comprising primary contiguous sequences within the structural-variation reference genome. As previously indicated, unlike the structural-variant-alignment tag 302 that indicates the alignment of nucleotide reads with an SV alternate contiguous sequence that does not exist in a linear reference genome or standard human reference genome (e.g., GRCh38), the split-alignment tag 304 indicates an alignment of a nucleotide read with a primary contiguous sequence that is part of such a linear reference genome or such a standard human reference genome. As shown in FIG. 3, the split-alignment tag 304 includes a reference identifier, an offset position, a strand direction identifier, a CIGAR, a mapping quality score, and an edit score. But the fields of the split-alignment tag 304 refer to the primary contiguous sequence of the structural-variation reference genome and/or the other flank of the nucleotide read. For example, unlike the CIGAR of the structural-variant-alignment tag, the CIGAR of the split-alignment tag refers to the primary contiguous sequence of the structural-variation reference genome. The split-alignment tag 304 accordingly uses a different reference point for mapping, offset, alignment, haplotype, editing, and other information than the disclosed structural-variant-alignment tag 302.

As mentioned previously, in some embodiments, the structural-variant-aware sequencing system 106 determines a candidate structural variant location for a target genomic region. In accordance with one or more embodiments, FIGS. 4A and 4B illustrate the structural-variant-aware sequencing system 106 determining genomic candidate coordinates for structural variants and generating a variant call format (VCF) file.

As shown in FIG. 4A, for instance, the structural-variant-aware sequencing system 106 identifies or receives nucleotide reads 402. In particular, the structural-variant-aware sequencing system 106 identifies one or more nucleotide reads corresponding to a genomic region of a genomic sample. In some cases, for instance, the structural-variant-aware sequencing system 106 receives base-call data (e.g., BCL file or FASTQ file) from a sequencing device. In some such cases, the base-call data takes the form of a base-call-data file that organizes single-end reads or paired-end reads according to index sequences attached to oligonucleotides extracted from a genomic sample. As indicated above, the structural-variant-aware sequencing system 106 can sequence or analyze short nucleotide reads (e.g., <300 base pairs or <10,000 base pairs) as the one or more nucleotide reads 506, in some implementations, or long nucleotide reads (e.g., >300 base pairs or >10,000 base pairs) as the one or more nucleotide reads 506, in other implementations. Such long nucleotide reads may include, for example, assembled nucleotide reads, CCS reads, or nanopore long reads.

As further shown in FIG. 4A, the structural-variant-aware sequencing system 106 aligns the nucleotide reads 402 with different sequences within the structural variation reference genome 404. For instance, the structural-variant-aware sequencing system 106 aligns subsets of nucleotide reads 406a and 406c in whole or in part with primary contiguous sequences 408a and 408b, respectively, of a linear reference genome 413. As indicated above, each of the primary contiguous sequences 408a-408b represent portions of a primary assembly within a structural variant reference genome, such as primary contiguous sequences from GRCh38. As further shown in FIG. 4A, the structural-variant-aware sequencing system 106 aligns a subset of nucleotide reads 406b in whole or in part with an alternate contiguous sequence 410 representing a structural variant haplotype (e.g., an insertion or a deletion of a threshold number of base pairs) or with an additional alternate contiguous sequence 411 representing an additional structural variant haplotype. As shown, in some cases, a fragment of a nucleotide read from the subset of nucleotide reads 406b aligns with the alternate contiguous sequence 410 or with the additional alternate contiguous sequence 411. By contrast, a full length of a nucleotide read from the subset of nucleotide reads 406b aligns completely within the alternate contiguous sequence 410.

For illustrative purposes and space constraints, FIG. 4A depicts the subsets of nucleotide reads 406a-406c, the primary contiguous sequences 408a-408b, the alternate contiguous sequence 410, and the additional alternate contiguous sequence 411, as merely examples. As indicated above, a sequencing device may generate numerous additional nucleotide reads, and the structural variation reference genome 404 may include numerous other types of primary contiguous sequences, alternate nucleobases, and/or alternate contiguous sequences. Indeed, the structural variation reference genome 404 depicted in FIG. 4A is merely one illustration to visualize a primary contiguous sequence and an alternate contiguous sequence of a structural variation reference genome embodied by a hash table, matrix, or other digital organizational structure. As explained further below, the additional alternate contiguous sequence 411 both (i) is optionally included or excluded within a structural-variation reference genome and (ii) represents an additional structural variant haplotype that differs from the structural variant haplotype represented by the alternate contiguous sequence 410. For instance, the additional alternate contiguous sequence 411 may represent an insertion or a deletion of a threshold number of base pairs, but longer or shorter than the structural variant haplotype represented by the alternate contiguous sequence 410, or an insertion or deletion as part of a structural variant haplotype comprising flanking variants (e.g., flanking SNVs) that differ from the structural variant haplotype represented by the alternate contiguous sequence 410.

Based on the identified nucleotide reads, the structural-variant-aware sequencing system 106 can determine if (i) one or more candidate alignment(s) between one or more nucleotide reads from the subsets of nucleotide reads 406a-406c and the alternate contiguous sequence 410 are more accurate than (ii) the candidate alignment(s) of the one or more nucleotide reads with the primary contiguous sequence. For example, the structural-variant-aware sequencing system 106 can determine if the candidate alignment of the alternate contiguous sequence 410 is better aligned than the candidate alignment of the same nucleotide reads with the primary contiguous sequences 408a or 408b. For example, to compare the candidate alignments of the nucleotides reads with the alternate contiguous sequence 410 and primary contiguous sequences 408a and 408b, the structural-variant-aware sequencing system 106 generates contiguity-aware alignment scores for the candidate alignment of each nucleotide read from the subsets of nucleotide reads 406a-406c with the corresponding primary contiguous sequence and, if relevant, the alternate contiguous sequence 410 or the additional alternate contiguous sequence 411.

As mentioned above, the structural-variant-aware sequencing system 106 generates contiguity-aware alignment scores for the alignment of one or more nucleotide reads with the primary contiguous sequence and an SV alternate contiguous sequence. In some cases, the contiguity-aware alignment scores comprise pair scores for paired end reads. As indicated above, in one or more implementations, a pair score is a composite score summing alignment scores for each mate and applying a pairing penalty, break penalty, and/or overlap penalty to the nucleotide reads. For example, the structural-variant-aware sequencing system 106 can generate a first pair score for candidate pair alignments between (i) a first candidate pair of split groups with nucleotide read fragments aligning and (ii) at least a part of a primary contiguous sequence (e.g., the primary contiguous sequence 408a or 408b). Moreover, the structural-variant-aware sequencing system 106 can generate a second pair score evaluating candidate pair alignments between (i) a second candidate pair of split groups comprising one or more nucleotide read fragments aligning and (ii) at least a part of an SV alternate contiguous sequence (e.g., the alternate contiguous sequence 410 or the additional alternate contiguous sequence 411). As indicated above, in some embodiments, the structural-variant-aware sequencing system 106 does not include a pairing penalty as part of the second pair score for a pair of candidate split groups with a candidate fragment alignment of a read fragment (or entire nucleotide read) with at least part of an SV alternate contiguous sequence (e.g., the alternate contiguous sequence 410).

In addition to first and second pair scores for candidate split groups, the structural-variant-aware sequencing system 106 can further generate pair scores for additional candidate split groups. Based on the second pair score being the highest pair score among the candidate pairs of split groups, such as when the second pair score exhibits a highest pair score among candidate split groups, the structural-variant-aware sequencing system 106 can generate an alignment file with the structural-variant-alignment tag.

As just mentioned, the structural-variant-aware sequencing system 106 can generate pair scores (e.g., hundreds or thousands of pair scores) for additional candidate split groups corresponding to paired-end reads. For example, nucleotide fragments of candidate split groups can have various alignment combinations with the structural-variation reference genome. For instance, a candidate split group comprising a first read of nucleotide fragment alignments (e.g., R1) and a second read of nucleotide fragment alignments (e.g., R2) can have nucleotide fragment alignments that (i) wholly align with the structural-variation reference genome, (ii) overlap, (iii) span a breakpoint, (iv) align with the alternate contiguous sequence representing the structural variant haplotype, or (v) have different orientations with respect to the structural-variation reference genome. In such cases, the structural-variant-aware sequencing system 106 can generate pair scores for each candidate split group corresponding to paired-end reads (e.g., R1 and R2) with respect to the primary contiguous sequence of structural-variation reference genome and/or the alternate contiguous sequence representing the structural variant haplotype. For example, the structural-variant-aware sequencing system 106 can generate a pair score for read fragments of a paired-end read aligned with one alternate contiguous sequence representing a structural variant haplotype and generate a different pair score for different read fragments of the paired-end read aligned with another alternate contiguous sequence representing a different structural variant haplotype.

Consistent with the disclosure above, in some embodiments, when a pair score for a candidate pair of split groups-which comprises a read fragment aligning with at least a part of an SV alternate contiguous sequence-exhibits a highest pair score among candidate split groups, the structural-variant-aware sequencing system 106 generates an alignment file with a structural-variant-alignment tag corresponding to a nucleotide read as part of the candidate pair of split groups. Such a candidate pair of split groups may exhibit a highest pair score from among hundreds (or more) of competing candidate pairs of split groups for a paired-end read.

In one or more embodiments, the structural-variant-aware sequencing system 106 can also determine a split group score for a split alignment of the fragments of one or more nucleotide reads among the subset of nucleotide reads 406b with the primary contiguous sequence shown with a liftover relationship with the alternate contiguous sequence 410, the additional alternate contiguous sequence 411, and/or other primary or alternate contiguous sequences within the structural-variation reference genome. For example, if the split group score for a split alignment of fragments of a nucleotide read among the nucleotide reads 406b and the alternate contiguous sequence 410 exceeds the first contiguity-aware alignment score for candidate alignments with the primary contiguous sequence and/or other alternate candidate alignments, the structural-variant-aware sequencing system 106 can generate the alignment file 414 with a corresponding structural-variant-alignment tag. In one or more embodiments, the structural-variant-aware sequencing system 106 can generate the second contiguity-aware alignment score by determining an alt-contig fragment alignment score for the alignment of a nucleotide read with the alternate contiguous sequence. In such a circumstance, the alt-contig fragment alignment score functions as a pair score for the relevant candidate split group. In some embodiments, the structural-variant-aware sequencing system 106 determines alt-contig fragment alignment scores, pair scores, and split group scores as described by Improving Split-Read Alignment by Intelligently Identifying and Scoring Candidate Split Groups, U.S. Patent Application No. 63/367,002 (filed Jun. 24, 2022), which is hereby incorporated by reference in its entirety.

As further shown in FIG. 4A, in one or more embodiments, the structural-variant-aware sequencing system 106 utilizes an imputation model 412 to determine a likelihood that the genomic sample includes a particular structural variant haplotype. Specifically, the structural-variant-aware sequencing system 106 inputs data representing an aligned set of nucleotide reads from the nucleotide reads 402 (e.g., nucleotide reads aligned with or overlapping a candidate genomic coordinate for structural variants) into the imputation model 412 to determine whether the genomic sample exhibits an additional structural variant haplotype represented by the additional alternate contiguous sequence 411. For example, in some instances, the nucleotide reads 402 identified from the genomic sample may include not only split-reads that span a breakpoint, such as a breakpoint 409 for the additional alternate contiguous sequence 411, but also flanking nucleotide reads that align in whole or in part with the additional alternate contiguous sequence 411. In these or other embodiments, the structural-variant-aware sequencing system 106 can determine a likelihood that the genomic sample includes the additional structural variant haplotype represented by the additional alternate contiguous sequence 411 using the imputation model 412 to process data representing an aligned set of nucleotide reads, including, but not limited to, such flanking nucleotide reads.

As just mentioned, in one or more embodiments, the structural-variant-aware sequencing system 106 utilizes the imputation model 412 to process data representing flanking nucleotide reads and/or other nucleotide reads of a sample to determine a likelihood that the genomic sample exhibits or comprises an additional structural variant haplotype. To illustrate, the structural-variant-aware sequencing system 106 may identify a flanking nucleotide read 407 that aligns to a genomic region of the additional alternate contiguous sequence 411 adjacent to, but not overlapping with, the breakpoint 409 within the linear reference genome 413. Although not depicted in FIG. 4A, the nucleotide reads 402 may include tens, hundreds, or thousands of such flanking reads within a threshold number of nucleobases (e.g., 50; 100; 1,000; 3,0000) of the breakpoint 409 of the linear reference genome 413. As indicated above, the breakpoint 409 of the linear reference genome 413 marks a genomic coordinate at which nucleotide reads (or fragments of nucleotides reads) split or align with different locations of the linear reference genome 413. Further, in one or more embodiments, the structural-variant-aware sequencing system 106 utilizes the imputation model 412 to determine that the flanking nucleotide read 407 (and/or other flanking nucleotide reads) includes a variant (e.g., an SNV or other variant) within the additional alternate contiguous sequence 411.

In addition to or part of determining the flanking nucleotide read 407 includes such a variant, the structural-variant-aware sequencing system 106 can utilize an HMM or other imputation-based model as the imputation model 412 to infer, from the variant of the flanking nucleotide read 407 and/or other variants exhibited by additional flanking nucleotide reads, a likelihood that the genomic sample includes the additional structural variant haplotype represented by the additional alternate contiguous sequence 411. For example, the structural-variant-aware sequencing system 106 can utilize the imputation model 412 to determine that (i) the variant of the flanking nucleotide read 407 and/or a variant of other flanking nucleotide reads is within the additional alternate contiguous sequence 411 representing the additional structural variant haplotype and (ii) the flanking nucleotide read 407 and/or other flanking nucleotide reads exhibit higher contiguity-aware alignment scores for candidate alignments with the additional alternate contiguous sequence 411 representing the additional structural variant haplotype than other, competing contiguity-aware alignment scores.

As just mentioned, the structural-variant-aware sequencing system 106 can use the imputation model 412 to determine that the variant of the flanking nucleotide read 407 is exhibited by the additional structural variant haplotype. Specifically, the structural-variant-aware sequencing system 106 generates contiguity-aware alignment scores for both the candidate alignment of the flanking nucleotide read 407 with the primary contiguous sequence and the candidate alignment with a genomic region of the additional alternate contiguous sequence 411 adjacent to the breakpoint 409. Based on the contiguity-aware alignment score for the candidate alignment of the flanking nucleotide read 407 with the additional alternate contiguous sequence 411 exceeding other contiguity-aware alignment scores, the structural-variant-aware sequencing system 106 can generate an alignment file with a structural-variant-alignment tag corresponding to the additional alternate contiguous sequence 411 associated with the flanking nucleotide read 407.

Similarly, in one or more embodiments, the structural-variant-aware sequencing system 106 may determine that the flanking nucleotide read 407 aligns better (i.e., has a higher contiguity-aware alignment score) with multiple, other alternate contiguous sequences (e.g., alternate contiguous sequence 410) than with the primary contiguous sequence. When a contiguity-aware alignment score for the flanking nucleotide read 407 is the same for candidate alignments with multiple alternate contiguous sequences, the mapping quality of the flanking nucleotide read 407 degrades. For instance, in some embodiments, the structural-variant-aware sequencing system 106 can generate the alignment file to include each of the alternate contiguous sequences with a corresponding MAPQ field value of zero and a structural-variant-alignment tag.

Having identified the additional alternate contiguous sequence 411 and/or other additional alternate contiguous sequences based on imputation, the structural-variant-aware sequencing system 106 can utilize such additional alternate contiguous sequences as candidates for identifying genomic coordinates with candidate structural variants. In one or more embodiments, for instance, the structural-variant-aware sequencing system 106 re-aligns a set of nucleotide reads from the nucleotide reads 402 with one or more alternate contiguous sequences of the structural variation reference genome after identifying additional alternate contiguous sequences using the imputation model 412. In these or other embodiments, the structural-variant-aware sequencing system 106 can limit or otherwise restrict the number of alternate contiguous sequences (i) included within a structural-variation reference genome and/or (ii) used for the re-alignment based on the initial alignment of nucleotide reads with the structural-variation reference genome. For example, the structural-variant-aware sequencing system 106 re-aligns such nucleotide reads with the primary contiguous sequence and one or more alternate contiguous sequences representing one or more structural variant haplotypes identified as most likely present in a genomic sample via the initial alignment and/or the alternate contiguous sequences identified via the imputation model 412. Further, in these or other embodiments, the structural-variant-aware sequencing system 106 generates the alignment file 414 with appropriate structural-variant-alignment tags based on the re-alignment.

As just mentioned, in one or more embodiments, the structural-variant-aware sequencing system 106 generates the alignment file 414 with appropriate structural-variant-alignment tags based on the re-alignment. Specifically, the structural-variant-aware sequencing system 106 generates contiguity-aware alignment scores for the nucleotide reads aligned with the primary contiguous sequence and the alternate contiguous sequences (e.g., alternate contiguous sequence 410 and additional alternate contiguous sequence 411). In these or other embodiments, the structural-variant-aware sequencing system 106 generates the alignment file 414 based on the contiguity-aware alignment scores as described above. For example, based on determining a highest contiguity-aware alignment score of a nucleotide read aligned with one or more alternate contiguous sequences, the structural-variant-aware sequencing system 106 generates the alignment file 414 to include one or more structural-variant-alignment tags corresponding to the nucleotide read.

In addition or in the alternative to identifying one or more alternate contiguous sequences as candidates for inclusion within a structural-variation reference genome, in one or more embodiments, the structural-variant-aware sequencing system 106 can utilize the imputation model 412 to exclude alternate contiguous sequences as candidates for the structural-variation reference genome. In particular, the structural-variant-aware sequencing system 106 can exclude alternate contiguous sequences (e.g., the additional alternate contiguous sequence 411) for re-alignment of one or more nucleotide reads of the nucleotide reads 402. For instance, the structural-variant-aware sequencing system 106 can utilize the imputation model 412 to process data representing an aligned set of nucleotide reads of a genomic sample (e.g., nucleotide reads aligned with or overlapping a candidate genomic coordinate for structural variants) and determine that an aligned set of nucleotide reads indicates that a likelihood that the genomic sample comprises a particular structural variant haplotype (e.g., the structural variant haplotype represented by the additional alternate contiguous sequence 411). In some cases, the imputation model 412 outputs such a likelihood, and the structural-variant-aware sequencing system 106 further determines that the likelihood does not satisfy a candidate-likelihood threshold. To further illustrate, in these or other embodiments, the structural-variant-aware sequencing system 106 excludes, based on the likelihood not satisfying the candidate-likelihood threshold, the additional alternate contiguous sequence 411 at the candidate genomic coordinate for re-alignment of the nucleotide reads.

As just suggested, in some embodiments, the structural-variant-aware sequencing system 106 can determine, utilizing the imputation model 412 to process data representing an aligned set of nucleotide reads, a first likelihood that a genomic sample comprises a structural variant haplotype represented by the alternate contiguous sequence 410 or a second likelihood that the genomic sample comprises an additional structural variant haplotype represented by the additional alternate contiguous sequence 411. Based on the first likelihood or the second likelihood, the structural-variant-aware sequencing system 106 re-aligns one or more nucleotide reads from the nucleotide reads 402 with one or more of the alternate contiguous sequence 410 at the candidate genomic coordinate, the additional alternate contiguous sequence 411 at the candidate genomic coordinate, or a corresponding primary contiguous sequence within the linear reference genome 413 at the candidate genomic coordinate.

As further shown in FIG. 4A, the structural-variant-aware sequencing system 106 generates an alignment file 414. In particular, the structural-variant-aware sequencing system 106 can generate the alignment file 414 comprising annotations indicating information about a structural variant haplotype detected in the genomic sample. For instance, after scanning the entire genome sequencing data set to detect candidate genomic coordinates for candidate structural variants, the structural-variant-aware sequencing system 106 identifies nucleotide reads exhibiting abnormal alignments or structural-variant-alignment tags for candidate structural-variant locations. To illustrate, the alignment file 414 can comprise a SAM file that includes the structural-variant-alignment tag mapping the nucleotide reads to genomic coordinates on the alternate contiguous sequence representing the structural variant haplotype. For example, the structural-variant-alignment tag for a particular nucleotide read can indicate candidate genomic coordinates for a candidate structural variant within a genomic sample. This disclosure describes one or more examples of such a structural-variant-alignment tag above with respect to FIG. 3.

To illustrate the utility of a structural-variant-alignment tag, in certain cases, one or more nucleotide reads from a genomic sample may span a breakpoint while aligned to a primary contiguous sequence. In some such instances, the alternate contiguous sequence may comprise a sequence that has been inserted or deleted at the breakpoint resulting in a nucleotide read more accurately aligning with an alternate contiguous sequence (e.g., the alternate contiguous sequence 410). Based on the contiguity-aware alignment scoring described above, the structural-variant-aware sequencing system 106 can generate a corresponding structural-variant-alignment tag that identifies the alternate contiguous sequence exhibits better alignment with the corresponding nucleotide read and the location of the nucleotide read with regard to the alternate contiguous sequence. In some cases, as shown in FIG. 4A, based on the structural-variant-alignment tag, the structural-variant-aware sequencing system 106 can determine candidate genomic coordinates for candidate structural variants 416.

As just mentioned, in some embodiments, the structural-variant-aware sequencing system 106 determines candidate genomic coordinates for candidate structural variants 416 by identifying nucleotide reads exhibiting abnormal alignments or structural-variant-alignment tags. As indicated above, the structural-variant-aware sequencing system 106 can identify nucleotide reads exhibiting abnormal alignments by identifying a cluster of nucleotide read alignments with masked fragments or pairs of read fragment (e.g., partial read) alignments falling below or exceeding a threshold insert size. In some embodiments, the threshold insert size is fixed. In alternative embodiments, the threshold insert size is dynamic and/or based on a model (e.g., mixed Gaussian model or other distribution model).

To illustrate, in some embodiments, the structural-variant-aware sequencing system 106 identifies a cluster of nucleotide read alignments with masked fragments or nucleobases that satisfy or exceed a threshold number of nucleobases (e.g., 15, 20, 35 nucleobases for each read in the cluster of nucleotide read alignments). In some cases, the nucleotide reads from the cluster include masked fragments that align with an alternate contiguous sequence. In one or more embodiments, the structural-variant-aware sequencing system 106 identifies a genomic coordinate for a corresponding primary contiguous sequence as a candidate genomic coordinate for a candidate structural variant.

Additionally, in some cases, the structural-variant-aware sequencing system 106 determines that nucleotide reads exhibit abnormal alignments by identifying nucleotide reads with an estimated or predicted insert size falling below or exceeding a threshold insert size. As mentioned above, the threshold insert size can be dynamic and change based on the dataset and/or the genomic sample.

To illustrate, in a read data set for a given genomic sample, the structural-variant-aware sequencing system 106 determines a distribution of insert sizes corresponding to paired-end reads by (i) modeling a distribution of insert sizes corresponding to paired-end reads of a genomic sample according to an Independent and Identically Distributed (IID) normal distribution (or other distribution model) and (ii) determining a standard deviation for the distribution of insert sizes. Based on the distribution of insert sizes and the standard deviation, the structural-variant-aware sequencing system 106 can identify insert sizes in an alignment file (e.g., SAM file) that fall outside of the standard deviation. When, for instance, the structural-variant-aware sequencing system 106 determines a mean insert size of 500 base pairs (or nucleobases) for a genomic sample with a standard deviation of 100 base pairs (or nucleobases), the structural-variant-aware sequencing system 106 identifies, from the genomic sample's alignment file, a determined insert size for paired-end reads outside of the standard deviation—at only 100 base pairs or at 1000 base pairs, for example—as falling below or exceeding a threshold insert size.

As mentioned above, the threshold insert size can be dynamic. More specifically, in some embodiments, the threshold insert size can consider (i) the size distribution of a genomic fragment from a sample library fragment and (ii) the actual insert size of the genomic fragment corresponding to paired-end reads. For example, in certain cases, the structural-variant-aware sequencing system 106 fits the actual insert size to a fitting model (e.g., Gaussian distribution or mixed Gaussian distribution). Based on the fitting model, the structural-variant-aware sequencing system 106 can identify pairs of nucleotide read fragment alignments with an insert size that fall below or exceed the threshold insert size. For example, if the expected fragment size distribution is 500 base pairs ±100 base pairs, nucleotide read fragment alignments with an insert size of 600 base pair fragments do not exceed the threshold insert size. Conversely, if the expected fragment size distribution is 250 base pairs ±50, a pair of read fragments with an insert size of 600 base pairs would exceed the threshold insert size, indicate an abnormal alignment, and provide support for a structural variant haplotype.

As further indicated by FIG. 4A, in one or more embodiments, the structural-variant-aware sequencing system 106 utilizes the imputation model 412 to determine candidate genomic coordinates for candidate structural variants 416. For example, the structural-variant-aware sequencing system 106 can determine that a flanking nucleotide read supports a candidate genomic coordinate of the candidate genomic coordinates for the structural variants corresponding to the nucleotide reads exhibiting the structural-variant-alignment tags using the imputation model 412.

The structural-variant-aware sequencing system 106 can, therefore, count or determine that a flanking nucleotide read constitutes a supporting nucleotide read for either a particular structural variant or for a genomic coordinate as a location for a candidate structural variant. To illustrate, in some instances, the structural-variant-aware sequencing system 106 identifies the flanking nucleotide read 407 (or another flanking nucleotide read) and determines that the flanking nucleotide read 407 (or another flanking nucleotide read) includes a variant exhibited by the additional alternate contiguous sequence 411, as described above. Having determine the flanking nucleotide read 407 exhibits such a variant, in some instances, the structural-variant-aware sequencing system 106 can use the imputation model 412 to identify a candidate genomic coordinate supported by the flanking nucleotide read 407 qualifies as a candidate genomic coordinate for a candidate structural variant.

Having determined that a candidate genomic coordinate supports a candidate genomic coordinate for a structural variant corresponding to the flanking nucleotide read 407 exhibiting a structural-variant-alignment tag in the alignment file 414, the structural-variant-aware sequencing system 106 can include the flanking nucleotide read 407 (or another such flanking nucleotide read with a structural-variant-alignment tag) among a subset of nucleotide reads for filtering. As described in further detail below, the structural-variant-aware sequencing system 106 can filter nucleotide reads corresponding to candidate genomic coordinates for candidate structural variants.

In addition or in the alternative to including such flanking nucleotide reads corresponding to candidate genomic coordinates for filtering, the structural-variant-aware sequencing system 106 can also identify a candidate genomic coordinate for a candidate structural variant based on imputation and independent of whether one or more of the nucleotide reads 402 aligns best with a particular alternate contiguous sequence. For instance, in some embodiments, the structural-variant-aware sequencing system 106 inputs data representing an aligned set of nucleotide reads from the nucleotide reads 402 into the imputation model 412; determines, utilizing the imputation model 412, a likelihood that a genomic sample exhibits the additional structural variant haplotype represented by the additional alternate contiguous sequence 411 and that such a likelihood exceeds or otherwise satisfies a candidate-likelihood threshold—even if no such flanking nucleotide read (e.g., the flanking nucleotide read 407) exhibits a highest contiguity-aware alignment score for a candidate alignment with the additional alternate contiguous sequence 411. Based on the likelihoods output by the imputation model 412—and despite the highest contiguity-aware alignment scores for flanking reads or other nucleotide reads with a particular alternate contiguous sequence—the structural-variant-aware sequencing system 106 can identify an alternate contiguous sequence representing a structural variant haplotype and a corresponding genomic coordinate as a candidate structural variant exhibited by a genomic sample.

As mentioned above, the structural-variant-aware sequencing system 106 can utilize structural-variant-alignment tags and/or abnormal alignments in the alignment file 414 to determine candidate genomic coordinates for candidate structural variants of a genomic sample. As shown in FIG. 4B, the structural-variant-aware sequencing system 106 generates a variant call file 426 by filtering and assembling nucleotide reads supporting candidate genomic coordinates for structural variants and scoring candidate structural variants of a genomic sample at such genomic coordinates in accordance with one or more embodiments.

As illustrated in FIG. 4B, the structural-variant-aware sequencing system 106 filters the nucleotide reads 420 corresponding to candidate genomic coordinates for candidate structural variants. In particular, the structural-variant-aware sequencing system 106 identifies a filtered set of nucleotide reads that (i) satisfy one or more quality metrics for a given candidate genomic coordinate from the candidate genomic coordinates and/or (ii) exhibit structural-variant-alignment tags, as described above. In particular, the quality metrics comprise the nucleotide reads passing filtering conditions and meeting quality criteria. For instance, in some embodiments, passing filtering conditions includes identifying a subset of nucleotide reads exhibiting a threshold mapping quality score or a specified flag status.

As just mentioned, in some cases, the structural-variant-aware sequencing system 106 selects identified nucleotide reads satisfying a threshold mapping quality score for further analysis. For instance, the threshold mapping quality (e.g., threshold MAPQ) score sets a minimum for correctly mapping nucleotide reads to either a primary contiguous sequence or an alternate contiguous sequence of a structural-variation reference genome. For a given genomic sample, a threshold mapping quality score can depend on the mapping of nucleotide reads and, therefore, differ from genomic sample to genomic sample. For instance, in some embodiments, the threshold mapping quality score is 30 for a given genomic sample and any mapping quality score meeting or exceeding MAPQ 30 satisfies this particular quality metric.

As previously indicated, in some embodiments, the structural-variant-aware sequencing system 106 can identify nucleotide reads that satisfy a quality metric by identifying nucleotide reads that exhibit a specified flag status. For instance, as discussed above the flag status (e.g., FLAG) in an alignment file is a combination of bitwise flags providing read alignment information in the SAM file. In particular, the flag status utilizes integers to represent twelve attributes/descriptions of the alignment of the nucleotide reads with either a primary contiguous sequence or an alternate contiguous sequence within a reference genome. For instance, the twelve attributes describe whether the nucleotide read is a paired read, part of properly aligned pair, unmapped, part of a pair and its mate was not mapped, aligned in the reverse direction relative to the structural-variation reference genome, part of a pair and its mate is aligned in the reverse direction relative to the structural-variation reference genome, the first read in the pair, the second read in pair, is note a primary alignment (e.g., secondary alignment), fails a platform quality check, a duplicate (e.g., optical duplicate, PCR duplicate, etc.), and/or supplementary alignment. In some cases, the structural-variant-aware sequencing system 106 removes (or filters out) nucleotide reads for which the alignment file comprises a duplicate flag status, a secondary alignment flag status, and/or fails the platform quality check flag status.

In addition or in the alternative to the quality metrics described above, the structural-variant-aware sequencing system 106 can select nucleotide reads supporting a candidate genomic coordinate based on other quality metrics. For instance, the nucleotide reads can meet quality metrics by exhibiting: a threshold number of nucleobases that have not been masked and that differ from one or more nucleobases of the primary contiguous sequence, a threshold insert size, and/or a CIGAR indicating an insertion operation or deletion operation.

As just mentioned, in some cases, the structural-variant-aware sequencing system 106 determines if a nucleotide read overlapping with a candidate genomic coordinate meets a quality metric by exhibiting a threshold number of nucleobases that have not been masked (e.g., not soft clipped) and that differ from one or more nucleobases of the primary contiguous sequence. For instance, the structural-variant-aware sequencing system 106 identifies nucleotide reads comprising unmasked nucleobases that exceed a threshold number of nucleobases (e.g., 10, 20, 30 nucleobases) that mismatch the aligned primary contiguous sequence. In such cases, the corresponding nucleotide reads will be kept for consideration of a structural variant haplotype for future analysis (e.g., assembly, scoring, structural variant calling). In some embodiments, the threshold number of nucleotide bases that have not been masked and differ from the nucleobases of the primary contiguous sequence is dynamic.

As previously indicated, the structural-variant-aware sequencing system 106 determines if a nucleotide read corresponding to a candidate genomic coordinate satisfies a quality metric by determining whether the nucleotide read exhibits a threshold insert size. As described above, exceeding a threshold insert size indicates abnormal alignments for nucleotide reads. Likewise, in some cases, the nucleotide reads exhibiting the threshold insert size (e.g., falling within a standard deviation of a mean insert size) satisfy a particular quality metric to be part of a filtered set of nucleotide reads.

Additionally, in some cases, the structural-variant-aware sequencing system 106 determines if a nucleotide read overlapping with a candidate genomic coordinate satisfies a quality metric by exhibiting a CIGAR indicating an insertion operation or a deletion operation with respect to either a primary contiguous sequence or an alternate contiguous sequence. As mentioned above, in one or more cases, the CIGAR provides an alignment summary for a given nucleotide read. In particular embodiments, the CIGAR indicates matches, deletions, and/or insertions between the nucleotide reads and either a primary contiguous sequence or an alternate contiguous sequence within the structural-variation reference genome. In some embodiments, a nucleotide read is selected as part of a filtered set of nucleotide reads when the CIGAR for the nucleotide read indicates a deletion and/or insertion.

As introduced above, the structural-variant-aware sequencing system 106 determines if a nucleotide read overlapping with a candidate genomic coordinate satisfies a quality metric by determining whether the nucleotide read exhibits a corresponding structural-variant-alignment tag in the alignment file. For instance, the structural-variant-aware sequencing system 106 can identify nucleotide reads in a SAM file that include a corresponding structural-variant-alignment tag. In some embodiments, regardless of the mapping quality score and/or flag status, the structural-variant-aware sequencing system 106 selects a nucleotide read as part of the filtered set of nucleotide reads when the nucleotide read has a corresponding structural-variant-alignment tag. By contrast, in some embodiments, a nucleotide read must also satisfy one or more other quality metrics to be part of the filtered set of nucleotide reads.

Relatedly, in addition or in the alternative to the quality metrics described above, the structural-variant-aware sequencing system 106 determines if a nucleotide read overlapping with a candidate genomic coordinate satisfies a quality metric by determining whether the nucleotide read exhibits a corresponding split alignment tag in the alignment file. For instance, the structural-variant-aware sequencing system 106 can identify nucleotide reads in a SAM file that include a corresponding split alignment tag. In some embodiments, regardless of the mapping quality score and/or flag status, the structural-variant-aware sequencing system 106 selects a nucleotide read as part of the filtered set of nucleotide reads when the nucleotide read has a corresponding split alignment tag. By contrast, in some embodiments, a nucleotide read must also satisfy one or more other quality metrics to be part of the filtered set of nucleotide reads.

As just discussed, the structural-variant-aware sequencing system 106 can identify a filtered set of nucleotide reads that satisfy one or more quality metrics at a candidate genomic coordinate corresponding to the target genomic region. As further illustrated in FIG. 4B, the structural-variant-aware sequencing system 106 assembles a contiguous nucleotide sequence 422 from the filtered set of nucleotide reads and/or from an imputed alternate contiguous sequence. In particular, the structural-variant-aware sequencing system 106 can assemble, from the filtered set of nucleotide reads supporting a given candidate structural-variant location, the contiguous sequence representing the structural variant haplotype exhibited by a genomic sample. For example, the structural-variant-aware sequencing system 106 can determine a consensus sequence representing a filtered set of nucleotide reads overlapping with a candidate genomic coordinate, where the consensus sequence represents a structural variant haplotype spanning a breakpoint or other marker. More specifically, in some implementations, the structural-variant-aware sequencing system 106 can assemble the contiguous nucleotide sequence 422 representing the structural variant haplotype exhibited by a genomic sample within the target genomic region by utilizing a reference-guided assembler tool to build a De Brujin graph (e.g., utilizing a De Brujin Graph (DBG) assembler) or inferring a consensus nucleotide sequence (e.g., utilizing an Overlap Layout Consensus (OLC) assembler as a reference-guided assembler tool).

To illustrate, in some embodiments, the structural-variant-aware sequencing system 106 utilizes a reference-guided assembler tool to assemble structural variant haplotypes at candidate genomic coordinates, for example, by building a local De Brujin graph using a DBG assembler or a consensus nucleotide sequence using an OLC assembler. In one or more implementations, the reference-guided assembler tool utilizes the primary contiguous sequence of the structural-variation reference genome as the backbone of the local De Brujin graph or consensus nucleotide sequence.

After assembling the contiguous nucleotide sequence, in some cases, the structural-variant-aware sequencing system 106 replaces a primary contiguous sequence with the contiguous nucleotide sequence 422 representing the structural variant haplotype. By substituting in the contiguous nucleotide sequence 422, the structural-variant-aware sequencing system 106 identifies a higher quality reference for structural variant scoring for candidate structural variant haplotypes within the target genomic region. More specifically, in cases where the structural-variant-aware sequencing system 106 replaces the primary contiguous sequence of structural-variation reference genome (e.g., GRCh38) with the contiguous nucleotide sequence 422, the structural-variant-aware sequencing system 106 can include k-mer nodes from the primary contiguous sequence and the contiguous nucleotide sequence 422 while building the local De Brujin graph or consensus nucleotide sequence. In such implementations, by building the local De Brujin graph or consensus nucleotide sequence with the k-mer nodes from the primary contiguous sequence and the contiguous nucleotide sequence 422, the structural-variant-aware sequencing system 106 can extract multiple high scoring paths representing structural variant haplotypes exhibited by the genomic sample within the target genomic region.

To further illustrate, in one or more embodiments, the reference-guided assembler tool utilizes an alternate contiguous sequence as a backbone of the local De Brujin graph or consensus nucleotide sequence rather than the primary contiguous sequence. For example, the structural-variant-aware sequencing system 106 can identify an alternate contiguous sequence, such as alternate contiguous sequence 410 or additional alternate contiguous sequence 411, as a reference-guide read 423. In these or other embodiments, the structural-variant-aware sequencing system 106 utilizes the reference-guided assembler tool to assemble the contiguous nucleotide sequence 422 from the filtered set of nucleotide reads and the reference-guide read 423 (e.g., the backbone). In the alternative to assembling the contiguous nucleotide sequence 422 from the filtered set of nucleotide reads, in some embodiments, the structural-variant-aware sequencing system 106 utilizes an alternate contiguous sequence identified based on likelihoods output by the imputation model 412 as the contiguous nucleotide sequence. After assembling or identifying the contiguous nucleotide sequence, in one or more embodiments, the structural-variant-aware sequencing system 106 can use the contiguous nucleotide sequence as a higher quality reference for structural variant scoring for candidate structural variant haplotypes within the target genomic region.

As previously discussed, the structural-variant-aware sequencing system 106 can additionally or alternatively assemble the given filtered set of nucleotide reads supporting the candidate genomic coordinates into the contiguous nucleotide sequence 422. In some embodiments, where the structural variant haplotype spans a breakpoint, the structural-variant-aware sequencing system 106 can assemble nucleotide reads supporting the candidate genomic coordinates at multiple locations. For example, in cases where the structural variant haplotype spans the breakpoint, the candidate genomic coordinates can be a pair of genomic coordinates. In such cases, the structural-variant-aware sequencing system 106 assembles the nucleotide reads supporting the candidate genomic coordinates from either side of the breakpoint. Moreover, in certain cases, the structural-variant-aware sequencing system 106 can represent the candidate genomic pairs as {(chr, pos), (chr, pos)}. In one or more embodiments, the representation of the candidate genomic pairs can be refined to them to {(chr, pos, orientation), (chr, pos, orientation)}.

As just mentioned, the structural-variant-aware sequencing system 106 can assemble nucleotide reads supporting the candidate genomic coordinates for candidate structural variants. In some embodiments, the structural-variant-aware sequencing system 106 identifies nucleotide reads within a genomic area flanking the candidate genomic coordinates for the structural variants. For example, in certain cases, after the structural-variant-aware sequencing system 106 identifies the candidate genomic coordinates, the structural-variant-aware sequencing system 106 can include nucleotide reads within a threshold distance from the genomic coordinate by assembling the nucleotide reads flanking the candidate genomic coordinates. Having identified such nucleotide reads within a threshold distance, the structural-variant-aware sequencing system 106 can assemble a given filtered set of nucleotide reads into the contiguous nucleotide sequence 422 representing the structural variant haplotype exhibited by the genomic sample within the target genomic region.

As further illustrated in FIG. 4B, the structural-variant-aware sequencing system 106 can generate structural variant scores 424 for candidate structural variants. In particular, the structural-variant-aware sequencing system 106 can generate, for the genomic sample at the target genomic region, one or more structural variant scores 424 for a candidate structural variant call. In some embodiments, the structural-variant-aware sequencing system 106 generates the structural variant scores 424 based on an allele frequency corresponding to the structural variant haplotype and alignment of an identified nucleotide read with the contiguous nucleotide sequence (e.g., the contiguous nucleotide sequence 422) or the corresponding primary contiguous sequence. For instance, the structural-variant-aware sequencing system 106 incorporates the allele frequency corresponding to the structural variant haplotype into a likelihood function (e.g., diploid scoring model).

As further shown in FIG. 4B, in one or more embodiments, the structural-variant-aware sequencing system 106 generates the structural variant scores 424 for a structural variant call based on a filtered set of nucleotide reads and an alternate contiguous sequence identified by a likelihood output by the imputation model 412. In particular, the structural-variant-aware sequencing system 106 can generate, for the genomic sample at the target genomic region, one or more structural variant scores 424 for a candidate structural variant haplotype identified via the imputation model 412. Specifically, the structural-variant-aware sequencing system 106 can identify candidate structural variant haplotypes associated with each alternate contiguous sequence identified via the imputation model 412 as described above.

To illustrate, the structural-variant-aware sequencing system 106 identifies a candidate structural variant haplotype represented by the additional alternate contiguous sequence 411. In some embodiments, the structural-variant-aware sequencing system 106 generates the structural variant scores 424 based on an allele frequency corresponding to the structural variant haplotype represented by the additional alternate contiguous sequence 411 and alignment of one or more identified nucleotide reads aligned with the additional alternate contiguous sequence 411 or the corresponding primary contiguous sequence. For instance, the structural-variant-aware sequencing system 106 incorporates the allele frequency corresponding to the structural variant haplotype represented by the additional alternate contiguous sequence 411 into a likelihood function (e.g., diploid scoring model).

As just suggested, in some embodiments, the structural-variant-aware sequencing system 106 can determine, utilizing the imputation model 412 to process data representing an aligned set of nucleotide reads, a first likelihood that a genomic sample comprises a structural variant haplotype represented by the alternate contiguous sequence 410 or a second likelihood that the genomic sample comprises an additional structural variant haplotype represented by the additional alternate contiguous sequence 411. Based on the first likelihood or the second likelihood, the structural-variant-aware sequencing system 106 one or more structural variant scores of the structural variant scores 424 for a candidate structural variant of the candidate structural variants based on an allele frequency corresponding to the structural variant haplotype represented by the alternate contiguous sequence 410 or the additional structural variant haplotype represented by the additional alternate contiguous sequence 411.

As just mentioned, in some embodiments, the structural-variant-aware sequencing system 106 generates structural variant scores 424 based on incorporating the allele frequency corresponding to the structural variant haplotype. In particular, the structural-variant-aware sequencing system 106 can score a candidate structural variant call by utilizing a diploid scoring model. For instance, the diploid scoring model generates diploid genotype probabilities for each candidate structural variant at a given candidate genomic coordinate. In some embodiments, for scoring purposes, the structural-variant-aware sequencing system 106 approximates the candidate structural variants based on alleles exhibited within the filtered set of nucleotide reads. In such cases, the structural-variant-aware sequencing system 106 applies a model with a single alternate allele.

For example, for reference and alternate alleles A={r, x}, where A represents possible combinations of an allele r representing a reference allele and x representing an alternate allele, the structural-variant-aware sequencing system 106 can apply a diploid scoring model for candidate genotypes that include or exclude alleles for a structural variant at a candidate genomic coordinate. In some cases, the structural-variant-aware sequencing system 106 restricts the genotypes for each allele to G={rr, rx, xx}, where G represents the genotype, rr represents alleles for a homozygous genotype of reference alleles from a primary contiguous sequence, rx represents alleles for a heterozygous genotype of one reference allele and one alternate allele from alternate contiguous sequence, and xx represents alleles with a homozygous genotype of alternate alleles from an alternate contiguous sequence (e.g., representing a structural variant).

As mentioned above, in some embodiments, the structural-variant-aware sequencing system 106 can generate the structural variant score by generating a posterior probability for a candidate genotype based on a prior probability for the candidate genotype. In one or more embodiments, the structural-variant-aware sequencing system 106 solves the posterior probability over G according to the following equation: P(G|D)∝P(D|G)P(G), where D represents all the supporting read fragments (or supporting nucleotide reads) for either allele, P(G|D) represents the posterior probability for a genotype given the supporting read fragments (or supporting nucleotide reads) for either allele, P(D|G) represents the probability of the supporting read fragments (or supporting nucleotide reads) for either allele given the genotype, and P(G) represents the prior probability for the genotype. Such supporting read fragments or supporting nucleotide reads can come from the filtered set of nucleotide reads. In one or more embodiments, the structural-variant-aware sequencing system 106 determines the prior probability P(G) according to the following equation:

$$P ( G ) = \{ \begin{matrix}{θ}_{SV} & if rx \\ {θ}_{SV} / 2 & if xx \\ 1 - {θ}_{SV} 3 / 2 & if rr\end{matrix}$$

where θ_{SV} represents an allele frequency for a structural variant heterozygosity. In some embodiments, the allele frequency for structural variant heterozygosity is set at a default of 1×10⁻⁵. By contrast, in some embodiments, the structural-variant-aware sequencing system 106 (i) identifies, from a haplotype database (e.g., Structural Variation Data Hub from the National Institutes of Health) or other population data, an allele frequency for a candidate structural variant haplotype represented by a given alternate contiguous sequence and (ii) uses the identified allele frequency for the candidate structural variant haplotype as θ_{SV} for structural variant heterozygosity. In one or more implementations, the structural-variant-aware sequencing system 106 computes the likelihood P(D|G) by assuming that each supporting read fragment (or supporting nucleotide read) represents an independent observation of the genomic sample. For instance, in some embodiments, the structural-variant-aware sequencing system 106 determines P(D|G) according to the following equation:

$$P ( D \vert G ) = {\prod }_{d ∈ D} P ( d \vert G )$$

where d∈D represents an independent observation of the supporting read fragments (or supporting nucleotide reads) of the genomic sample and P(d|G) represents a read fragment likelihood (or nucleotide read likelihood). As further indicated below in some embodiments, the structural-variant-aware sequencing system 106 determines the fragment likelihood (or nucleotide read likelihood) by summing together the read fragments (or nucleotide reads) according to the following equation:

$$P ( d \vert G ) = {\sum }_{a ∈ A} P ( d \vert a ) P ( a \vert G )$$

where a∈A represents an independent observation of the alleles, P(d|a) represents a likelihood for each read fragment (or nucleotide read) to support the given allele, and P(a|G) represents standard diploid variant frequencies from {0,0.5,1}.

To determine a structural variant call for a target genomic region of a genomic sample, the structural-variant-aware sequencing system 106 determines a genotype call based on the structural variant scores (e.g., posterior genotype probabilities). For instance, in certain implementations, the structural-variant-aware sequencing system 106 selects a genotype exhibiting a highest posterior genotype probability (among genotype probabilities) for a target genomic region or genomic coordinate as the genotype call for a genomic sample. If the selected genotype exhibits a structural variant, the structural-variant-aware sequencing system 106 generates a structural variant call for the genomic sample at the target genomic region or the target genomic coordinate. Accordingly, the structural-variant-aware sequencing system 106 can generate, for a target genomic region of a genomic sample, a structural variant call based on a candidate alignment of the one or more nucleotide reads with at least part of an alternate contiguous sequence representing a structural variant haplotype.

After the structural-variant-aware sequencing system 106 generates structural variant scores and determines genotype calls and/or structural variant calls, in some embodiments, the structural-variant-aware sequencing system 106 generates a variant call file 426. As discussed above, the variant call file 426 refers to a digital file that indicates and/or represents one or more reference calls and variant calls for a genomic sample, including any structural variant calls. For instance, in some cases, the structural-variant-aware sequencing system 106 generates the variant call file 426 comprising (i) an annotation indicating one or more variant calls represents a structural variant haplotype and/or (ii) a structural-variant-alignment tag indicating an alignment reflecting the structural variant haplotype within the genomic sample. Consistent with the disclosure above, a given variant call can correspond to a structural variant haplotype comprising a deletion of more than a threshold number of base pairs, an insertion of more than the threshold number of base pairs, a duplication of more than the threshold number of base pairs, an inversion, a translocation, or a copy number variation (CNV).

Relatedly, the structural-variant-aware sequencing system 106 can generate structural variant calls corresponding to structural variant haplotypes based on nucleotide alignments with the structural variation reference genome. In particular, based on the alignment of the subset of nucleotide reads 406b and the alternate contiguous sequence 410, for example, the structural-variant-aware sequencing system 106 generates one or more structural variant calls indicating the target genomic region of the genomic sample exhibits the structural variant haplotype represented by the alternate contiguous sequence 410. As discussed above, the variant call file 426 can include data indicating the structural variant call based on the alignment of the nucleotide reads with at least a part of the alternate contiguous sequence.

As discussed above, the structural-variant-aware sequencing system 106 can determine if one or more nucleotide reads align partially or completely within the alternate contiguous sequence representing a structural variant haplotype. In accordance with one or more embodiments, FIGS. 5A-5C illustrate the structural-variant-aware sequencing system 106 aligning and masking nucleotide reads with respect to an alternate contiguous sequence representing a structural variant haplotype. As shown in FIGS. 5A-5C, nucleotide reads with a dashed line indicate a read pair.

FIG. 5A shows one or more nucleotide reads 506 aligning with a primary contiguous sequence 502 and an alternate contiguous sequence 504 representing a structural variant haplotype. In particular, FIG. 5A illustrates the alternate contiguous sequence 504 representing a deletion event where the alignment of the one or more nucleotide reads 506 with the alternate contiguous sequence 504 exhibits a better contiguity-aware alignment score than the alignment of one or more nucleotide reads 506 with the primary contiguous sequence 502. For instance, as shown in FIG. 5A, the structural-variant-aware sequencing system 106 aligns a corresponding nucleotide read 510 with the following nucleotide base sequence ACATCCGC to the sequence of nucleotide bases 508 (e.g., ACATCCGC) on the primary contiguous sequence 502. As illustrated in FIG. 5A, the alignment of the corresponding nucleotide read 510 with the primary contiguous sequence 502 spans a breakpoint. In particular, during the alignment of the corresponding nucleotide read 510 with the primary contiguous sequence 502, there is a breakpoint (e.g., break) at structural variant length (“SVLEN”) 512 that separates nucleobases AC from nucleobases ATCCGC resulting in a fragment alignment at one location on the primary contiguous sequence 502 (e.g., before SVLEN 512) and another fragment alignment on the primary contiguous sequence (e.g., after SVLEN 512).

As further shown in FIG. 5A, the structural-variant-aware sequencing system 106 aligns the one or more nucleotide reads 506 (including the corresponding nucleotide read 510) with the alternate contiguous sequence 504. For instance, as illustrated in FIG. 5A, by aligning the one or more nucleotide reads 506 with the alternate contiguous sequence 504 corresponding to the breakpoint, a break no longer exists in the corresponding nucleotide read 510. For example, in some embodiments, the alignment of the corresponding nucleotide read 510 with the alternate contiguous sequence 504 creates a full-length alignment on the alternate contiguous sequence 504. In such cases, a contiguity-aware alignment score for (i) a candidate alignment between the corresponding nucleotide read 510 and the alternate contiguous sequence 504 is higher than (ii) a candidate alignment between the corresponding nucleotide read 510 and the primary contiguous sequence 502 because the contiguity-aware alignment score for (i) (e.g., second contiguity-aware alignment score) is not penalized by the breakpoint.

As just indicated, the structural-variant-aware sequencing system 106 determines that the contiguity-aware alignment score for the candidate alignment between the corresponding nucleotide read 510 and the alternate contiguous sequence 504 exceeds the contiguity-aware alignment score (e.g., first contiguity-aware alignment score) for the candidate alignment between the corresponding nucleotide read 510 and the primary contiguous sequence 502. As discussed above, in some cases, when the second contiguity-aware alignment score for the alternate contiguous sequence 504 exceeds the first contiguity-aware alignment score for the primary contiguous sequence 502, such as when the second contiguity-aware alignment score exceeds other contiguity-aware alignment scores, the structural-variant-aware sequencing system 106 generates a structural-variant-alignment tag within an alignment file for the corresponding nucleotide read 510.

As indicated in FIG. 5A and discussed above, when the structural-variant-aware sequencing system 106 determines that the candidate alignment of a nucleotide read from the one or more nucleotide reads 506 with the alternate contiguous sequence 504 exhibits a higher contiguity-aware alignment score than the candidate alignment of the nucleotide read from the one or more nucleotide reads 506 with the primary contiguous sequence 502, the structural-variant-aware sequencing system 106 generates a corresponding structural-variant-alignment tag. Conversely, in some cases, where the candidate alignment of a nucleotide read from the one or more of nucleotide reads 506 with the alternate contiguous sequence 504 exhibits a lower contiguity-aware alignment score than the candidate alignment of the nucleotide read from the one or more nucleotide reads 506 with the primary contiguous sequence 502, the structural-variant-aware sequencing system 106 does not generate a corresponding structural-variant-alignment tag. For instance, in cases where nucleotide reads do not span a breakpoint on the primary contiguous sequence 502, the candidate alignment of the nucleotide reads with the alternate contiguous sequence 504 will not improve the contiguity-aware alignment score because the nucleotide reads are not penalized by the breakpoint.

FIG. 5B illustrates an embodiment of the structural-variant-aware sequencing system 106 determining that the candidate alignment of a set of nucleotide reads 522 with the alternate contiguous sequence 504 is not better scoring than the candidate alignment of the set of nucleotide reads 522 with the primary contiguous sequence 502. More specifically, as shown in FIG. 5B, the set of nucleotide reads 522 do not include a corresponding nucleotide read 524 aligning with the primary contiguous sequence 502. When the set of nucleotide reads 522 do not have a corresponding nucleotide read 524 aligned with the primary contiguous sequence 502, the structural-variant-aware sequencing system 106 will not generate a corresponding structural-variant-alignment tag.

As mentioned above, the structural-variant-aware sequencing system 106 can utilize the breakpoint and/or liftover relationship between a primary contiguous sequence and an alternate contiguous sequence to align nucleotide reads with the alternate contiguous sequence and identify a genomic coordinate or region for such an alignment with respect to a primary contiguous sequence. In accordance with one or more embodiments, FIG. 5C illustrates the structural-variant-aware sequencing system 106 masking and/or aligning nucleotide reads for various insertion events by utilizing the breakpoint and/or the liftover relationship.

As shown in FIG. 5C, in some cases, the structural-variant-aware sequencing system 106 determines that one or more nucleotide reads 536 overlaps with an alternate contiguous sequence 532 representing the structural variant haplotype. In particular, the structural-variant-aware sequencing system 106 determines if one or more of the nucleotide reads 536 span a breakpoint at SVLEN 534 within the primary contiguous sequence 530, where the alternate contiguous sequence 532 corresponds to the breakpoint for the SVLEN 534. For example, one or more of the nucleotide reads 536 and/or corresponding read fragments partially overlap with a portion of the primary contiguous sequence 530 and a portion of the alternate contiguous sequence 532 representing an insertion. In such cases, the structural-variant-aware sequencing system 106 can mask (e.g., soft-clip) the insertion-overlapping nucleotide reads and/or corresponding read fragments aligned with the alternate contiguous sequence 532 representing the insertion.

As mentioned above, however, due to sequence duplications, homologues, and other similar sequence content within a reference genome, a sequencing system can map nucleotide reads and/or corresponding read fragments to multiple different genomic regions along a reference genome, such as multiple different primary contiguous sequences. Because multiple such mappings can exhibit low or near zero mapping quality and existing sequencing systems lack a model to account for ambiguous mappings of low quality, existing sequencing systems can leave such ambiguously mapped nucleotide reads unresolved (e.g., no calls). Despite such mapping ambiguities, about a candidate alignment of a nucleotide read or read fragment with a structural variant haplotype for an insertion may be correct, but nevertheless be mapped to split over two different locations and lead to confusion about the actual location of an insertion event.

As discussed above, the structural-variant-aware sequencing system 106 avoids such ambiguities by aligning the nucleotide reads and/or fragments of the nucleotide reads aligned with the alternate contiguous sequence 532 representing an insertion of a threshold number of base pairs to a corresponding genomic coordinate on the primary contiguous sequence 530. In particular, the structural-variant-aware sequencing system 106 can guide unmasked nucleotide reads and/or unmasked fragments of the nucleotide reads aligned to the corresponding location on the primary contiguous sequence 530 by utilizing the liftover relationship between the primary contiguous sequence 530 and the alternate contiguous sequence 532. In particular, the structural-variant-aware sequencing system 106 can utilize the presence of the structural-variant-alignment tag and the liftover relationship between the alternate contiguous sequence 532 and the primary contiguous sequence 530 to align the nucleotide reads and/or fragments of the nucleotide reads at a single, genomic coordinate corresponding to the primary contiguous sequence 530. For example, in some embodiments, based on the liftover relationship, the corresponding genomic coordinate on the primary contiguous sequence 530 is adjacent to the breakpoint on the primary contiguous sequence 530. For example, in some embodiments, the liftover relationship between the primary contiguous sequence 530 and alternate contiguous sequence 532 lifts over the nucleotide reads and/or nucleotide read fragments to a point adjacent to SVLEN 534. In one or more implementations, where the liftover relationship does not indicate the corresponding coordinate to the primary contiguous sequence 530, the structural-variant-aware sequencing system 106 aligns the nucleotide reads at the breakpoint coordinate on the primary contiguous sequence 530.

As just described, the liftover relationship between the primary contiguous sequence 530 and the alternate contiguous sequence 532 guides the structural-variant-aware sequencing system 106 to align one or more of the nucleotide reads 536 and/or corresponding read fragments with the alternate contiguous sequence 532 positioned at a genomic coordinate. Thus, in one or more implementations, based on the liftover relationship, the structural-variant-aware sequencing system 106 can generate a clean pile up of nucleotide reads 536 and/or fragments of the nucleotide reads 536 supporting a single nominal breakpoint position on the primary contiguous sequence 530. For instance, the structural-variant-aware sequencing system 106 can mask the nucleotide reads 536 and/or fragments of the nucleotide reads 536 aligned with the alternate contiguous sequence 532 and align the unmasked nucleotide reads 536 and/or fragments of the nucleotide reads 536 aligned with the primary contiguous sequence 530 to a single, corresponding genomic coordinate on the primary contiguous sequence 530 based on the liftover relationship.

As just discussed, the structural-variant-aware sequencing system 106 can align nucleotide reads 536 and/or fragments of the nucleotide reads 536 that partially overlap with the primary contiguous sequence 530 and the alternate contiguous sequence 532 representing an insertion with respect to the primary contiguous sequence 530. FIG. 5C further illustrates the structural-variant-aware sequencing system 106 identifying an insertion-marker genomic coordinate for the nucleotide reads 540 that completely align within the alternate contiguous sequence 532 representing the insertion. For example, the structural-variant-aware sequencing system 106 can determine that some nucleotide reads 540 are not represented in the primary contiguous sequence 530. More specifically, the structural-variant-aware sequencing system 106 can determine that the nucleotide reads 540 do not exhibit as high of a contiguity-aware alignment score for candidate alignments with primary contiguous sequences as with the alternate contiguous sequence 532.

In cases where the nucleotide reads 540 align completely within the alternate contiguous sequence 532 representing the insertion at SVLEN 534, the structural-variant-aware sequencing system 106 can identify an insertion-marker genomic coordinate at which the insertion is lifted over within the structural-variation reference genome. For instance, the structural-variant-aware sequencing system 106 may identify a genomic coordinate directly adjacent to the SVLEN 534 as the insertion-marker genomic coordinate.

To further illustrate such a marker, the structural-variant-aware sequencing system 106 can identify the insertion-marker genomic coordinate for the nucleotide reads 540 that completely align within the alternate contiguous sequence 532. In particular, the structural-variant-aware sequencing system 106 can identify the insertion-marker genomic coordinate by utilizing the liftover relationship between the primary contiguous sequence 530 and the alternate contiguous sequence 532. For example, based on the liftover relationship, the structural-variant-aware sequencing system 106 can lift over the insertion with respect to the insertion-marker genomic coordinate within a primary contiguous sequence (e.g., from CRGh38) within the structural-variation reference genome. For example, in one or more embodiments, the structural-variant-aware sequencing system 106 can lift over the first aligned base before the insertion at SVLEN 534 from the alternate contiguous sequence 532 to the breakpoint on the primary contiguous sequence 530. In some embodiments with multiple nucleotide reads 540 aligned completely within the alternate contiguous sequence 532 representing the insertion at SVLEN 534, the structural-variant-aware sequencing system 106 sorts the nucleotide reads 540 at the insertion-marker genomic coordinate based on alignment information contained in the structural-variant-alignment tag (e.g., alternate-sequence identifier, offset position, etc.). In some embodiments, where the one or more nucleotide reads 540 align with multiple locations on the alternate contiguous sequence 532, the liftover relationship between the alternate contiguous sequence 532 and the primary contiguous sequence 530 guides the one or more nucleotide reads 536 to the same genomic coordinate (e.g., insertion-marker genomic coordinate) on the primary contiguous sequence.

In some embodiments, after lifting over the insertion marked by SVLEN 534, the structural-variant-aware sequencing system 106 fully masks (e.g., completely hard or soft clips) nucleobases within the nucleotide reads 540. In particular, the structural-variant-aware sequencing system 106 can generate an alignment file with a masking indicator showing the nucleobases of the nucleotide reads 540 are fully masked. To illustrate, in the SAM file, the structural-variant-aware sequencing system 106 can indicate that the alignments of the nucleotide reads 540 are unmapped on the primary contiguous sequence 530. In some cases, the structural-variant-aware sequencing system 106 generates an unmapped identifier indicating the nucleotide read is not mapped to any primary contiguous sequence (e.g., the primary contiguous sequence 530) within the structural-variation reference genome. Additionally, in one or more embodiments, the structural-variant-aware sequencing system 106 can generate a completed-clipping identifier indicating that the nucleotide read is fully clipped or does not require fragment masking for alignment. In certain implementations, the presence of a pile-up of the nucleotide reads 540 at the insertion-marker genomic coordinate enables structural variant callers to detect the insertion at a genomic coordinate marked by SVLEN 534 because the nucleotide reads 540 can act as evidence of the insertion.

As mentioned above, in certain described embodiments, the structural-variant-aware sequencing system 106 improves the accuracy of variant calling over existing sequencing systems. In particular, the structural-variant-aware sequencing system 106 improves short alignment and structural variant haplotype detection and other variant detection. FIG. 6 illustrates bar graphs 600 of experiments demonstrating the accuracy and structural variant detection improvements of the structural-variant-aware sequencing system 106 as compared to a baseline sequencing system.

For reference and as depicted in FIG. 6, the name “baseline” refers to an existing sequencing system that utilizes a reference genome to detects structural variants. In particular, the baseline sequencing system cannot generate a structural-variant-alignment tag or mask nucleobases of nucleotide reads based on breakpoints or insertion-marker genomic coordinates, as provided by the structural-variant-aware sequencing system 106. The term “GraphHT” in FIG. 6 refers to an embodiment of the structural-variant-aware sequencing system 106 that generates structural-variant-alignment tag, masks nucleobases of nucleotide reads based on breakpoints or insertion-marker genomic coordinates, and determines structural variant scores (as described above) using a structural-variation reference genome in the form of a hash table.

As further depicted in FIG. 6, the term “EZ2Map” represents regions on the linear reference genome to which nucleotide reads are easy to map in terms of MAPQ. Moreover, the term “MHC” represents the major histocompatibility complex (MHC) regions on the reference genome. The term “nonTR” represents regions on the linear reference genome that are not inside of Tandem Repeat regions. Additionally, the term “SharedTP” represents regions on the linear reference genome that are shared by both a sample specific truth set and a structural variant set representing structural variant haplotypes as alternate contiguous sequences within the hash-table version of the structural variation reference genome. Finally, the term “WG” represents the whole genome.

As depicted in FIG. 6, the disclosed embodiment of the structural-variant-aware sequencing system 106 outperforms the baseline sequencing system in accurately calling variants across various genomic contexts. For instance, the bar graphs 600 depict improved F-score and recall values for detecting variants representing deletions (DEL) of a threshold number of base pairs (e.g., >50 base pairs) and insertions (INS) of a threshold number of base pairs (e.g., >50 base pairs). For instance, as shown in the bar graphs 600, the F-Score and recall score for deletions and insertions for regions on the linear reference genome that are difficult to map (“Diff2MaP”) is higher for the embodiments of the structural-variant-aware sequencing system 106 than in baseline sequencing system. Likewise, the embodiment of the structural-variant-aware sequencing system 106 improved the score and recall values for insertions and deletions at all tested regions on the linear reference genome (EZ2Map, MHC, nonTR, SharedTP, WG). To further illustrate, the embodiment of the structural-variant-aware sequencing system 106 has improved the accuracy and sensitivity of detecting structural variants across the entire genome region.

FIGS. 1-6, the corresponding text, and the examples provide a number of different methods, systems, devices, and non-transitory computer-readable media of the structural-variant-aware sequencing system 106. In addition to the foregoing, one or more implementations can also be described in terms of flowcharts comprising acts for accomplishing a particular result, as shown in FIG. 7 and FIG. 8.

Turning now to FIG. 7, this figure illustrates a flowchart of a series of acts 700 of generating a structural-variant-alignment tag in accordance with one or more embodiments of the present disclosure. While FIG. 7 illustrates acts according to one embodiment, alternative embodiments may omit, add to, reorder, and/or modify any of the acts shown in FIG. 7. The acts of FIG. 7 can be performed as part of a method. Alternatively, a non-transitory computer readable storage medium can comprise instructions that, when executed by one or more processors, cause a computing device or a system to perform the acts depicted in FIG. 7. In still further embodiments, a system comprising at least one processor and a non-transitory computer readable medium comprising instructions that, when executed by one or more processors, cause the system to perform the acts of FIG. 7. In some cases, the at least one processor comprises a configurable processor and executing the at least one processor comprises configuring the configurable processor.

As illustrated in FIG. 7, the series of acts 700 includes the act 702 of identifying nucleotide reads from a genomic sample. In particular, the structural-variant-aware sequencing system 106 identifies one or more nucleotide reads corresponding to a target genomic region of a genomic.

As further illustrated in FIG. 7, the structural-variant-aware sequencing system performs the act 704 of generating contiguity-aware alignment scores. More particularly, the structural-variant-aware sequencing system 106 generates a first contiguity-aware alignment score for an candidate alignment of the one or more nucleotide reads with at least part of a primary contiguous sequence of a structural-variation reference genome and a second contiguity-aware alignment score for an candidate alignment of the one or more nucleotide reads with at least part of an alternate contiguous sequence representing a structural variant haplotype within the structural-variation reference genome.

As FIG. 7 further illustrates, the acts 700 include an act 706 of generating an alignment filed comprising a structural-variant-alignment tag. In particular, in certain implementations, the act 706 includes generating, based on the second contiguity-aware alignment score exceeding the first contiguity-aware alignment score, an alignment file comprising a structural-variant-alignment tag indicating the candidate alignment of the one or more nucleotide reads with at least part of the alternate contiguous sequence.

As further shown in FIG. 7, the acts 700 include an act 708 of selecting a candidate structural-variant location based on the structural-variant-alignment tag. In particular, in certain implementations, the act 708 includes selecting the target genomic region as a candidate structural-variant location for variant calling based on the structural-variant-alignment tag.

In addition or in the alternative to the acts 710-740, in certain implementations, the acts 700 further include generating, for the target genomic region of the genomic sample, a structural variant call based on the candidate alignment of the one or more nucleotide reads with at least part of the alternate contiguous sequence.

As suggested above, in some embodiments, the acts 700 include generating the structural-variant-alignment tag comprising one or more of: an alternate-sequence identifier identifying the alternate contiguous sequence; an offset position for the one or more nucleotide reads with respect to the alternate contiguous sequence within the structural-variation reference genome; a strand-direction identifier for a forward strand or a reverse strand corresponding to the one or more nucleotide reads with respect to the alternate contiguous sequence; a concise idiosyncratic gapped alignment report (CIGAR) for the one or more nucleotide reads with respect to the alternate contiguous sequence; mapping quality score for a mapping of the one or more nucleotide reads to at least the alternate contiguous sequence; or an edit distance between nucleobases of the one or more nucleotide reads and the alternate contiguous sequence.

Additionally, in certain embodiments, the acts 700 include an act where the structural variant haplotype comprises a deletion of more than fifty base pairs, an insertion of more than fifty base pairs, a duplication of more than fifty base pairs, an inversion, a translocation, or a copy number variation (CNV). Moreover, in some cases, the acts 700 include an act where the structural variant haplotype comprises a deletion of more than twenty-five base pairs, an insertion of more than twenty-five base pairs, or a duplication of more than twenty-five base pairs.

In some cases, the acts include determining a fragment of a nucleotide read of the one or more nucleotide reads aligns with the alternate contiguous sequence representing an insertion; masking the fragment of the nucleotide read that aligns with the alternate contiguous sequence; and aligning an unmasked fragment of the nucleotide read with a given primary contiguous sequence of the structural-variation reference genome adjacent to a breakpoint for the alternate contiguous sequence.

In addition, one or more implementations, the acts 700 include determining a nucleotide read of the one or more nucleotide reads aligns completely within the alternate contiguous sequence representing an insertion of more than fifty base pairs; identifying, within a given primary contiguous sequence, an insertion-marker genomic coordinate at which the insertion is lifted over within the structural-variation reference genome; and generating the alignment file comprising an unaligned base indicator that the nucleotide read is fully masked with respect to the insertion-marker genomic coordinate.

In still some embodiments, the series of acts includes generating, within the alignment file, an unmapped identifier indicating the nucleotide read is not mapped to any primary contiguous sequence within the structural-variation reference genome; and generating, within the alignment file, a completed-clipping identifier indicating the nucleotide read is fully clipped or does not require fragment masking for alignment.

Moreover, in some implementations, the acts 700 include aligning a set of nucleotide reads of the genomic sample with a candidate genomic coordinate for structural variants; determining, utilizing an imputation model to process data representing the aligned set of nucleotide reads, a first likelihood that the genomic sample comprises the structural variant haplotype represented by the alternate contiguous sequence or a second likelihood that the genomic sample comprises an additional structural variant haplotype represented by an additional alternate contiguous sequence; and re-aligning, based on the first likelihood or the second likelihood, one or more nucleotide reads of the set of nucleotide reads with one or more of the alternate contiguous sequence at the candidate genomic coordinate, the additional alternate contiguous sequence at the candidate genomic coordinate, or the primary contiguous sequence at the candidate genomic coordinate.

In some cases, the acts 700 include determining the second likelihood does not satisfy a candidate-likelihood threshold; and excluding, based on the second likelihood not satisfying the candidate-likelihood threshold, the additional alternate contiguous sequence at the candidate genomic coordinate for re-alignment of one or more nucleotide reads of the set of nucleotide reads.

Additionally, in one or more embodiments, the series of acts 700 include identifying the alternate contiguous sequence as the reference-guide read; and assembling, utilizing a reference-guided assembler tool, the contiguous nucleotide sequence from the filtered set of nucleotide reads and the alternate contiguous sequence as the reference-guide read.

Further, in some implementations, the series of acts 700 includes determining the candidate genomic coordinates for the structural variants corresponding to the nucleotide reads exhibiting the structural-variant-alignment tags in part by: identifying, for the genomic sample, a flanking nucleotide read that aligns to a genomic region of the alternate contiguous sequence adjacent to a breakpoint for the alternate contiguous sequence; determining the flanking nucleotide read comprises a variant within the alternate contiguous sequence; and determining the flanking nucleotide read supports a candidate genomic coordinate of the candidate genomic coordinates for the structural variants corresponding to the nucleotide reads exhibiting the structural-variant-alignment tags.

Moreover, in one or more implementations, the series of acts 700 includes aligning a set of nucleotide reads of the genomic sample with a candidate genomic coordinate for structural variants; determining, utilizing an imputation model to process data representing the aligned set of nucleotide reads, a first likelihood that the genomic sample comprises the structural variant haplotype represented by the alternate contiguous sequence or a second likelihood that the genomic sample comprises an additional structural variant haplotype represented by an additional alternate contiguous sequence; and generating, for the genomic sample at the target genomic region and based on the first likelihood or the second likelihood, one or more structural variant scores for a structural variant call based on an allele frequency corresponding to the structural variant haplotype or the additional structural variant haplotype.

In certain implementations the acts 700 include identifying the one or more nucleotide reads by determining, for the target genomic region, candidate pairs of split groups for a pair of paired-end nucleotide reads; and generating the first contiguity-aware alignment score and the second contiguity-aware alignment score by: generating a first pair score evaluating pair alignments of a first candidate pair of split groups comprising one or more nucleotide read fragments aligning with at least part of the primary contiguous sequence; and generating a second pair score evaluating pair alignments of a second candidate pair of split groups comprising one or more nucleotide read fragments aligning with at least part of the alternate contiguous sequence.

In one or more cases, the acts 700 include determining the second contiguity-aware alignment score exceeds the first contiguity-aware alignment score by determining the second pair score exhibits a highest pair score among candidate pairs of split groups corresponding to the target genomic region; and generating the alignment file by generating the structural-variant-alignment tag indicating the pair alignments of the second candidate pair of split groups with the alternate contiguous sequence.

As mentioned above, FIG. 8 illustrates a flowchart of a series of acts 800 generating one or more structural variant scores for a structural variant. While FIG. 8 illustrates acts according to one implementation, alternative implementations may omit, add to, reorder, and/or modify any of the acts shown in FIG. 8. The acts of FIG. 8 can be performed as part of a method. Alternatively, a non-transitory computer-readable medium can comprise instructions that, when executed by at least one processor, cause a computing device to perform the acts of FIG. 8. In still further embodiments, a system comprising at least one processor and a non-transitory computer readable medium comprising instructions that, when executed by one or more processors, cause the system to perform the acts of FIG. 8. In some cases, the at least one processor comprises a configurable processor and executing the at least one processor comprises configuring the configurable processor.

As shown in FIG. 8, the series of acts 800 includes an act 802 of determining candidate genomic coordinates for structural variants for a genomic sample. In particular, the act 802 comprises determining, for the genomic sample, candidate genomic coordinates for structural variants corresponding to nucleotide reads exhibiting abnormal alignments or structural-variant-alignment tags.

The series of acts 800 illustrated in FIG. 8 further includes an act 804 of identifying a filtered set of nucleotide reads satisfying quality metrics and/or exhibiting structural-variant-alignment tags. In particular, the act 804 comprises identifying, at a candidate genomic coordinate corresponding to the target genomic region from among the candidate genomic coordinates, a filtered set of nucleotide reads that satisfy one or more quality metrics and/or exhibit one or more structural-variant-alignment tags.

As further shown in FIG. 8, the series of acts 800 includes an act 806 of assembling a contiguous nucleotide sequence representing a structural variant haplotype. In particular, the act 806 comprises assembling, from the filtered set of nucleotide reads, a contiguous nucleotide sequence representing the structural variant haplotype exhibited by the genomic sample within the target genomic region.

As FIG. 8 illustrates, the series of acts 800 includes and act 808 of generating one or more structural variant scores for candidate structural variants. In particular, the act 808 comprises generating, for the genomic sample at the target genomic region, one or more structural variant scores for candidate structural variant calls based on an allele frequency corresponding to the structural variant haplotype.

Additionally, or alternatively, the series of acts 802-808 include an act of determining the nucleotide reads exhibiting abnormal alignments by identifying a cluster of one or more nucleotide read alignments with masked fragments or pairs of read fragment alignments with an insert size falling below or exceeding a threshold insert size.

Moreover, in some embodiments, the series of acts 800 comprises identifying the filtered set of nucleotide reads that satisfy the one or more quality metrics by identifying a subset of nucleotide reads exhibiting one or more of: a threshold mapping quality score; a specified flag status; a corresponding structural-variant-alignment tag; a threshold number of nucleobases that have not been masked and that differ from one or more nucleobases of the primary contiguous sequence; a split alignment from a split-alignment tag; a threshold insert size; or a concise idiosyncratic gapped alignment report (CIGAR) indicating an insertion operation or a deletion operation.

In certain implementations, the series of acts 800 includes an act of replacing the primary contiguous sequence of the reference genome with the contiguous nucleotide sequence; and generating, for the genomic sample at the target genomic region, one or more structural variant scores for a structural variant call based on an allele frequency corresponding to the structural variant haplotype and candidate alignment of the one or more nucleotide reads with the contiguous nucleotide sequence or the primary contiguous sequence.

Moreover, in some implementations, the acts 800 include aligning a set of nucleotide reads of the genomic sample with a candidate genomic coordinate for structural variants; determining, utilizing an imputation model to process data representing the aligned set of nucleotide reads, a first likelihood that the genomic sample comprises the structural variant haplotype represented by the alternate contiguous sequence or a second likelihood that the genomic sample comprises an additional structural variant haplotype represented by an additional alternate contiguous sequence; and re-aligning, based on the first likelihood or the second likelihood, one or more nucleotide reads of the set of nucleotide reads with one or more of the alternate contiguous sequence at the candidate genomic coordinate, the additional alternate contiguous sequence at the candidate genomic coordinate, or the primary contiguous sequence at the candidate genomic coordinate.

In some cases, the acts 800 include determining the second likelihood does not satisfy a candidate-likelihood threshold; and excluding, based on the second likelihood not satisfying the candidate-likelihood threshold, the additional alternate contiguous sequence at the candidate genomic coordinate for re-alignment of one or more nucleotide reads of the set of nucleotide reads.

Additionally, in one or more embodiments, the series of acts 800 include identifying the alternate contiguous sequence as the reference-guide read; and assembling, utilizing a reference-guided assembler tool, the contiguous nucleotide sequence from the filtered set of nucleotide reads and the alternate contiguous sequence as the reference-guide read.

Further, in some implementations, the series of acts 800 includes determining the candidate genomic coordinates for the structural variants corresponding to the nucleotide reads exhibiting the structural-variant-alignment tags in part by: identifying, for the genomic sample, a flanking nucleotide read that aligns to a genomic region of the alternate contiguous sequence adjacent to a breakpoint for the alternate contiguous sequence; determining the flanking nucleotide read comprises a variant within the alternate contiguous sequence; and determining the flanking nucleotide read supports a candidate genomic coordinate of the candidate genomic coordinates for the structural variants corresponding to the nucleotide reads exhibiting the structural-variant-alignment tags.

Moreover, in one or more implementations, the series of acts 800 includes aligning a set of nucleotide reads of the genomic sample with a candidate genomic coordinate for structural variants; determining, utilizing an imputation model to process data representing the aligned set of nucleotide reads, a first likelihood that the genomic sample comprises the structural variant haplotype represented by the alternate contiguous sequence or a second likelihood that the genomic sample comprises an additional structural variant haplotype represented by an additional alternate contiguous sequence; and generating, for the genomic sample at the target genomic region and based on the first likelihood or the second likelihood, one or more structural variant scores for a structural variant call based on an allele frequency corresponding to the structural variant haplotype or the additional structural variant haplotype.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Implementations in which images are obtained in different color channels, for example, coinciding with different labels used to distinguish one nucleobase type from another are particularly applicable. In some implementations, the process to determine the nucleotide sequence of a target nucleic acid (i.e., a nucleic-acid polymer) can be an automated process. Preferred implementations include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as the release of pyrophosphate; or the like. In implementations, where two or more different nucleotides are present in a sequencing reagent, the different nucleotides can be distinguishable from each other, or alternatively, the two or more different labels can be indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred implementations include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to the incorporation of nucleotides at the features of the array. An image can be obtained after the array is treated with a particular nucleotide type (e.g., A, T, C, or G). Images obtained after the addition of each nucleotide type will differ with regard to which features in the array are detected. These differences in the image reflect the different sequence content of the features on the array. However, the relative locations of each feature will remain unchanged in the images. The images can be stored, processed, and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.) and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing implementations, the labels do not substantially inhibit extension under SBS reaction conditions. However, the detection labels can be removable, for example, by cleavage or degradation. Images can be captured following the incorporation of labels into arrayed nucleic acid features. In particular implementations, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such implementations, each image will show nucleic acid features that have incorporated nucleotides of a particular type. Different features are present or absent in the different images due to the different sequence content of each feature. However, the relative position of the features will remain unchanged in the images. Images obtained from such reversible terminator-SBS methods can be stored, processed, and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular implementations, some or all of the nucleotide monomers can include reversible terminators. In such implementations, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30-second exposure to long-wavelength UV light. Thus, either disulfide reduction or photocleavage can be used as a cleavable linker. Another approach to reversible termination is the use of natural termination that ensues after the placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some implementations can utilize the detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes an apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on the presence of their respective signals and incorporation of the fourth nucleotide type into the nucleic acid can be determined based on the absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary implementation that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some implementations can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due to the different sequence content of each feature, but the relative position of the features will remain unchanged in the images. Images obtained from ligation-based sequencing methods can be stored, processed, and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some implementations can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such implementations, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed, and analyzed as set forth herein. In particular, the data can be treated as an image in accordance with the exemplary treatment of optical images and other images that is set forth herein.

Some implementations can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed, and analyzed as set forth herein.

Some SBS implementations include the detection of a proton released upon incorporation of a nucleotide into an extension product. For example, sequencing based on detection of released protons can use an electrical detector and associated techniques that are commercially available from Ion Torrent (Guilford, C T, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular implementations, different target nucleic acids can be treated in a common reaction vessel or on a surface of a particular substrate. This allows convenient delivery of sequencing reagents, removal of unreacted reagents, and detection of incorporation events in a multiplex manner. In implementations using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle, or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for the detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing implementation as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, a “sample” (and its derivatives) is used in its broadest sense and includes any specimen, culture, and the like that is suspected of including a target. In some implementations, the sample comprises DNA, RNA, PNA, LNA, chimeric or hybrid forms of nucleic acids. The sample can include any biological, clinical, surgical, agricultural, atmospheric, or aquatic-based specimen containing one or more nucleic acids. The term also includes any isolated nucleic acid sample such a genomic DNA, fresh-frozen, or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample, and normal tissue sample, or sample from a single source that contains two distinct forms of genetic material such as maternal and fetal DNA obtained from a maternal subject, or the presence of contaminating bacterial DNA in a sample that contains plant or animal DNA. In some implementations, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another implementation, low molecular weight material includes enzymatically or mechanically fragmented DNA. The sample can include cell-free circulating DNA. In some implementations, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the sample can be an epidemiological, agricultural, forensic, or pathogenic sample. In some implementations, the sample can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one implementation, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example, derived from a buccal swab, paper, fabric, or other substrates that may be impregnated with saliva, blood, or other bodily fluids. As such, in some implementations, the nucleic acid sample may comprise low amounts of, or fragmented portions of DNA, such as genomic DNA. In some implementations, target sequences can be present in one or more bodily fluids including but not limited to, blood, sputum, plasma, semen, urine, and serum. In some implementations, target sequences can be obtained from hair, skin, tissue samples, autopsy or remains of a victim. In some implementations, nucleic acids including one or more target sequences can be obtained from a deceased animal or human. In some implementations, target sequences can include nucleic acids obtained from non-human DNA such a microbial, plant, or entomological DNA. In some implementations, target sequences or amplified target sequences are directed to purposes of human identification. In some implementations, the disclosure relates generally to methods for identifying characteristics of a forensic sample. In some implementations, the disclosure relates generally to human identification methods using one or more target-specific primers disclosed herein or one or more target-specific primers designed using the primer design criteria outlined herein. In one implementation, a forensic or human identification sample containing at least one target sequence can be amplified using any one or more of the target-specific primers disclosed herein or using the primer criteria outlined herein.

The components of the structural-variant-aware sequencing system 106 can include software, hardware, or both. For example, the components of the structural-variant-aware sequencing system 106 can include one or more instructions stored on a computer-readable storage medium and executable by processors of one or more computing devices (e.g., the client device 114). When executed by the one or more processors, the computer-executable instructions of the structural-variant-aware sequencing system 106 can cause the computing devices to perform the structural variant detection methods described herein. Alternatively, the components of the structural-variant-aware sequencing system 106 can comprise hardware, such as special-purpose processing devices to perform a certain function or group of functions. Additionally, or in the alternative, the components of the structural-variant-aware sequencing system 106 can include a combination of computer-executable instructions and hardware.

Furthermore, the components of the structural-variant-aware sequencing system 106 performing the functions described herein with respect to the structural-variant-aware sequencing system 106 may, for example, be implemented as part of a stand-alone application, as a module of an application, as a plug-in for applications, as a library function or functions that may be called by other applications, and/or as a cloud-computing model. Thus, components of the structural-variant-aware sequencing system 106 may be implemented as part of a stand-alone application on a personal computing device or a mobile device. Additionally, or alternatively, the components of the structural-variant-aware sequencing system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Implementations of the present disclosure may comprise or utilize a special purpose or general-purpose computer including computer hardware, such as, for example, one or more processors and system memory, as discussed in greater detail below. Implementations within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general-purpose or special-purpose computer system. Computer-readable media that store computer-executable instructions are non-transitory computer-readable storage media (devices). Computer-readable media that carry computer-executable instructions are transmission media. Thus, by way of example, and not limitation, implementations of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid-state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links that can be used to carry desired program code means in the form of computer-executable instructions or data structures and which can be accessed by a general-purpose or special-purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some implementations, computer-executable instructions are executed on a general-purpose computer to turn the general-purpose computer into a special-purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 9 illustrates a block diagram of a computing device 900 that may be configured to perform one or more of the processes described above. One will appreciate that one or more computing devices such as the computing device 900 may implement the structural-variant-aware sequencing system 106 and the sequencing device system 104. As shown by FIG. 9, the computing device 900 can comprise a processor 902, a memory 904, a storage device 906, an I/O interface 1008908 and a communication interface 910, which may be communicatively coupled by way of a communication infrastructure 912. In certain implementations, the computing device 900 can include fewer or more components than those shown in FIG. 9. The following paragraphs describe components of the computing device 900 shown in FIG. 9 in additional detail.

In one or more implementations, the processor 902 includes hardware for executing instructions, such as those making up a computer program. As an example, and not by way of limitation, to execute instructions for dynamically modifying workflows, the processor 902 may retrieve (or fetch) the instructions from an internal register, an internal cache, the memory 904, or the storage device 906 and decode and execute them. The memory 904 may be a volatile or non-volatile memory used for storing data, metadata, and programs for execution by the processor(s). The storage device 906 includes storage, such as a hard disk, flash disk drive, or another digital storage device, for storing data or instructions for performing the methods described herein.

The I/O interface 908 allows a user to provide input to, receive output from, and otherwise transfer data to and receive data from computing device 900. The I/O interface 908 may include a mouse, a keypad or a keyboard, a touch screen, a camera, an optical scanner, network interface, modem, other known I/O devices, or a combination of such I/O interfaces. The I/O interface 908 may include one or more devices for presenting output to a user, including, but not limited to, a graphics engine, a display (e.g., a display screen), one or more output drivers (e.g., display drivers), one or more audio speakers, and one or more audio drivers. In certain implementations, the I/O interface 908 is configured to provide graphical data to a display for presentation to a user. The graphical data may be representative of one or more graphical user interfaces and/or any other graphical content as may serve a particular implementation.

The communication interface 910 can include hardware, software, or both. In any event, the communication interface 910 can provide one or more interfaces for communication (such as, for example, packet-based communication) between the computing device 900 and one or more other computing devices or networks. As an example, and not by way of limitation, the communication interface 910 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 910 may facilitate communications with various types of wired or wireless networks. The communication interface 910 may also facilitate communications using various communication protocols. The communication infrastructure 912 may also include hardware, software, or both that couples components of the computing device 900 to each other. For example, the communication interface 910 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary implementations thereof. Various implementations and aspects of the present disclosure(s) are described with reference to details discussed herein, and the accompanying drawings illustrate the various implementations. The description above and drawings are illustrative of the disclosure and are not to be construed as limiting the disclosure. Numerous specific details are described to provide a thorough understanding of various implementations of the present disclosure.

The present disclosure may be embodied in other specific forms without departing from its spirit or essential characteristics. The described implementations are to be considered in all respects only as illustrative and not restrictive. For example, the methods described herein may be performed with fewer or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
identify one or more nucleotide reads corresponding to a target genomic region of a genomic sample;
generate a first contiguity-aware alignment score for a candidate alignment of the one or more nucleotide reads with at least part of a primary contiguous sequence of a structural-variation reference genome and a second contiguity-aware alignment score for a candidate alignment of the one or more nucleotide reads with at least part of an alternate contiguous sequence representing a structural variant haplotype within the structural-variation reference genome;
generate, based on the second contiguity-aware alignment score exceeding the first contiguity-aware alignment score, an alignment file comprising a structural-variant-alignment tag indicating the candidate alignment of the one or more nucleotide reads with at least part of the alternate contiguous sequence; and
select the target genomic region as a candidate structural-variant location for variant calling based on the structural-variant-alignment tag.

2. The system of claim 1, further comprising instructions that, when executed by the at least one processor, cause the system to generate, for the target genomic region of the genomic sample, a structural variant call based on the candidate alignment of the one or more nucleotide reads with at least part of the alternate contiguous sequence.

3. The system of claim 1, further comprising instructions that, when executed by the at least one processor, cause the system to generate the structural-variant-alignment tag comprising one or more of:
an alternate-sequence identifier identifying the alternate contiguous sequence;
an offset position for the one or more nucleotide reads with respect to the alternate contiguous sequence within the structural-variation reference genome;
a strand-direction identifier for a forward strand or a reverse strand corresponding to the one or more nucleotide reads with respect to the alternate contiguous sequence;
a concise idiosyncratic gapped alignment report (CIGAR) for the one or more nucleotide reads with respect to the alternate contiguous sequence;
a mapping quality score for a mapping of the one or more nucleotide reads to at least the alternate contiguous sequence; or
an edit distance between nucleobases of the one or more nucleotide reads and the alternate contiguous sequence.

4. The system of claim 1, wherein the structural variant haplotype comprises a deletion of more than fifty base pairs, an insertion of more than fifty base pairs, a duplication of more than fifty base pairs, an inversion, a translocation, or a copy number variation (CNV).

5. The system of claim 1, further comprising instructions that, when executed by the at least one processor, cause the system to:
determine a fragment of a nucleotide read of the one or more nucleotide reads aligns with the alternate contiguous sequence representing an insertion;
mask the fragment of the nucleotide read that aligns with the alternate contiguous sequence; and
align an unmasked fragment of the nucleotide read with a given primary contiguous sequence of the structural-variation reference genome adjacent to a breakpoint for the alternate contiguous sequence.

6. The system of claim 1, further comprising instructions that, when executed by the at least one processor, cause the system to:
determine a nucleotide read of the one or more nucleotide reads aligns completely within the alternate contiguous sequence representing an insertion of more than fifty base pairs;
identify, within a given primary contiguous sequence, an insertion-marker genomic coordinate at which the insertion is lifted over within the structural-variation reference genome; and
generate the alignment file comprising an unaligned read base indicator that the nucleotide read is fully masked with respect to the insertion-marker genomic coordinate.

7. The system of claim 6, further comprising instructions that, when executed by the at least one processor, cause the system to:
generate, within the alignment file, an unmapped identifier indicating the nucleotide read is not mapped to any primary contiguous sequence within the structural-variation reference genome; and
generate, within the alignment file, a completed-clipping identifier indicating the nucleotide read is fully clipped or does not require fragment masking for alignment.

8. The system of claim 1, further comprising instructions that, when executed by the at least one processor, cause the system to:
align a set of nucleotide reads of the genomic sample with a candidate genomic coordinate for structural variants;
determine, utilizing an imputation model to process data representing the aligned set of nucleotide reads, a first likelihood that the genomic sample comprises the structural variant haplotype represented by the alternate contiguous sequence or a second likelihood that the genomic sample comprises an additional structural variant haplotype represented by an additional alternate contiguous sequence; and
re-align, based on the first likelihood or the second likelihood, one or more nucleotide reads of the set of nucleotide reads with one or more of the alternate contiguous sequence at the candidate genomic coordinate, the additional alternate contiguous sequence at the candidate genomic coordinate, or the primary contiguous sequence at the candidate genomic coordinate.

9. The system of claim 8, further comprising instructions that, when executed by the at least one processor, cause the system to:
determine the second likelihood does not satisfy a candidate-likelihood threshold; and
exclude, based on the second likelihood not satisfying the candidate-likelihood threshold, the additional alternate contiguous sequence at the candidate genomic coordinate for re-alignment of one or more nucleotide reads of the set of nucleotide reads.

10. A non-transitory computer-readable medium comprising instructions that, when executed by at least one processor, cause a computing device to:
identify one or more nucleotide reads corresponding to a target genomic region of a genomic sample;
generate a first contiguity-aware alignment score for a candidate alignment of the one or more nucleotide reads with at least part of a primary contiguous sequence of a structural-variation reference genome and a second contiguity-aware alignment score for a candidate alignment of the one or more nucleotide reads with at least part of an alternate contiguous sequence representing a structural variant haplotype within the structural-variation reference genome;
generate, based on the second contiguity-aware alignment score exceeding the first contiguity-aware alignment score, an alignment file comprising a structural-variant-alignment tag indicating the candidate alignment of the one or more nucleotide reads with at least part of the alternate contiguous sequence; and
select the target genomic region as a candidate structural-variant location for variant calling based on the structural-variant-alignment tag.

11. The non-transitory computer-readable medium of claim 10, further comprising instructions that, when executed by the at least one processor, cause the computing device to:
determine, for the genomic sample, candidate genomic coordinates for structural variants corresponding to nucleotide reads exhibiting abnormal alignments or structural-variant-alignment tags;
identify, at a candidate genomic coordinate corresponding to the target genomic region from among the candidate genomic coordinates, a filtered set of nucleotide reads that satisfy one or more quality metrics and/or exhibit one or more structural-variant-alignment tags;
assemble, from the filtered set of nucleotide reads and/or a reference-guide read, a contiguous nucleotide sequence representing the structural variant haplotype exhibited by the genomic sample within the target genomic region; and
generate, for the genomic sample at the target genomic region, one or more structural variant scores for a structural variant call based on an allele frequency corresponding to the structural variant haplotype.

12. The non-transitory computer-readable medium of claim 11, further comprising instructions that, when executed by the at least one processor, cause the computing device to determine the nucleotide reads exhibiting abnormal alignments by identifying a cluster of one or more nucleotide read alignments with masked fragments or pairs of read fragment alignments with an insert size falling below or exceeding a threshold insert size.

13. The non-transitory computer-readable medium of claim 11, further comprising instructions that, when executed by the at least one processor, cause the computing device to identify the filtered set of nucleotide reads that satisfy the one or more quality metrics by identifying a subset of nucleotide reads exhibiting one or more of:
a threshold mapping quality score;
a specified flag status;
a corresponding structural-variant-alignment tag;
a threshold number of nucleobases that have not been masked and that differ from one or more nucleobases of the primary contiguous sequence;
a split alignment from a split-alignment tag;
a threshold insert size; or
a concise idiosyncratic gapped alignment report (CIGAR) indicating an insertion operation or a deletion operation.

14. The non-transitory computer-readable medium of claim 11, further comprising instructions that, when executed by the at least one processor, cause the computing device to:
replace the primary contiguous sequence of the structural-variation reference genome with the contiguous nucleotide sequence; and
generate, for the genomic sample at the target genomic region, the one or more structural variant scores for the structural variant call based on the allele frequency corresponding to the structural variant haplotype and alignment of the one or more nucleotide reads with the contiguous nucleotide sequence or the primary contiguous sequence.

15. The non-transitory computer-readable medium of claim 11, further comprising instructions that, when executed by the at least one processor, cause the computing device to assemble the contiguous nucleotide sequence by:
identifying the alternate contiguous sequence as the reference-guide read; and
assembling, utilizing a reference-guided assembler tool, the contiguous nucleotide sequence from the filtered set of nucleotide reads and the alternate contiguous sequence as the reference-guide read.

16. The non-transitory computer-readable medium of claim 11, further comprising instructions that, when executed by the at least one processor, cause the computing device to determine the candidate genomic coordinates for the structural variants corresponding to the nucleotide reads exhibiting the structural-variant-alignment tags in part by:
identifying, for the genomic sample, a flanking nucleotide read that aligns to a genomic region of the alternate contiguous sequence adjacent to a breakpoint for the alternate contiguous sequence;
determining the flanking nucleotide read comprises a variant within the alternate contiguous sequence; and
determining the flanking nucleotide read supports a candidate genomic coordinate of the candidate genomic coordinates for the structural variants corresponding to the nucleotide reads exhibiting the structural-variant-alignment tags.

17. A method comprising:
identifying one or more nucleotide reads corresponding to a target genomic region of a genomic sample;
generating a first contiguity-aware alignment score for a candidate alignment of the one or more nucleotide reads with at least part of a primary contiguous sequence of a structural-variation reference genome and a second contiguity-aware alignment score for a candidate alignment of the one or more nucleotide reads with at least part of an alternate contiguous sequence representing a structural variant haplotype within the structural-variation reference genome;
generating, based on the second contiguity-aware alignment score exceeding the first contiguity-aware alignment score, an alignment file comprising a structural-variant-alignment tag indicating the candidate alignment of the one or more nucleotide reads with at least part of the alternate contiguous sequence; and
selecting the target genomic region as a candidate structural-variant location for variant calling based on the structural-variant-alignment tag.

18. The method of claim 17, further comprising:
aligning a set of nucleotide reads of the genomic sample with a candidate genomic coordinate for structural variants;
determining, utilizing an imputation model to process data representing the aligned set of nucleotide reads, a first likelihood that the genomic sample comprises the structural variant haplotype represented by the alternate contiguous sequence or a second likelihood that the genomic sample comprises an additional structural variant haplotype represented by an additional alternate contiguous sequence; and
generating, for the genomic sample at the target genomic region and based on the first likelihood or the second likelihood, one or more structural variant scores for a structural variant call based on an allele frequency corresponding to the structural variant haplotype or the additional structural variant haplotype.

19. The method of claim 17, further comprising:
identifying the one or more nucleotide reads by determining, for the target genomic region, candidate pairs of split groups for a pair of paired-end nucleotide reads; and
generating the first contiguity-aware alignment score and the second contiguity-aware alignment score by:
generating a first pair score evaluating pair alignments of a first candidate pair of split groups comprising one or more nucleotide read fragments aligning with at least part of the primary contiguous sequence; and
generating a second pair score evaluating pair alignments of a second candidate pair of split groups comprising one or more nucleotide read fragments aligning with at least part of the alternate contiguous sequence.

20. The method of claim 19, further comprising:
determining the second contiguity-aware alignment score exceeds the first contiguity-aware alignment score by determining the second pair score exhibits a highest pair score among candidate pairs of split groups corresponding to the target genomic region; and
generating the alignment file by generating the structural-variant-alignment tag indicating the pair alignments of the second candidate pair of split groups with the alternate contiguous sequence.
